(19) United States
(12) Patent Application Publication
(10) Pub. No.: US 2006/0110401 A1
(43) Pub. Date: May 25, 2006

(54) Mixture of peptides from c and ns3 proteins of the hepatitis c virus and applications thereof

(76) Inventors: Bernard Maillere, Versailles (FR); Bertrand Georges, Bauvin (FR); Florence Castelli, Montrouge (FR); Ahmed Bouzidi, Annoeullin (FR)

Correspondence Address: MORGAN LEWIS & BOCKIUS LLP, 1111 PENNSYLVANIA AVENUE NW, WASHINGTON, DC, 20004, US

(21) Appl. No.: 10/522,992

(22) PCT Filed: Aug. 1, 2003
(86) PCT No.: PCT/FR03/02446

§ 371 Date: Oct. 11, 2005

(30) Foreign Application Priority Data
Aug. 2, 2002 (FR) 02/09874

(51) Int. Cl. A61K 39/29; C07K 14/18

(52) U.S. Cl. 424/189.1; 530/350

(57) ABSTRACT

The invention relates to a peptide mixture including at least two different peptides from the hepatitis C virus (HCV), whereby at least one of them is a peptide from the C protein binding to at least four different HLA-II molecules, the occurrence of which exceeds 5% in the Caucasian population with a binding activity <1,000 nM. Said invention also relates to the applications thereof as a drug useful in the prevention and treatment of HCV infections or as a diagnostic reagent for HCV-specific T lymphocytes.

The present invention relates to a mixture of peptides derived from the C and NS3 proteins of the hepatitis C virus (HCV) and also to applications thereof as a medicinal product (in immunogenic compositions capable of stimulating the production of anti-HCV CD4+ T lymphocytes in vivo and therefore useful for the prevention and treatment of HCV infections) or as a reagent for diagnosing HCV-specific T lymphocytes, in particular for evaluating the immune state of patients.

The hepatitis C virus (HCV) was identified in 1989 as being the major etiological agent of non-A, non-B hepatitis (Kato et al., P.N.A.S., 1990, 87, 9524-). Humans are the only known host of HCV, but the virus can be transmitted experimentally to chimpanzees. HCV infects hepatocytes, but also certain cells of the immune system such as lymphocytes, monocytes and dendritic cells.

Transmission of the virus is essentially parenteral. However, since de novo infections are rarely detectable, the route of contamination remains unknown in more than 40% of cases. The prevalence of hepatitis C is high in the general population, of the order of 1 to 2% in Africa, in America, in Europe and in South-East Asia (Alter et al., Blood, 1995, 85, 1681-). It could reach 5% in certain regions of China and in the west Pacific. In the Middle East, the prevalence varies from 1 up to 12%. Thus, it is estimated that 170 million individuals throughout the world are HCV carriers.

HCV is part of the Hepacivirus genus within the family Flaviviridae. It is a 9.4 kb positive-strand RNA virus. The genome has a main open reading frame which encodes a long polypeptide that can be cleaved into 8 proteins. The capsid protein (core or C, 191 amino acids) has the ability to bind to the viral RNA and constitutes the viral nucleocapsid. It also participates in direct cytopathogenic effects. The E1 and E2 glycoproteins are inserted into the viral envelope and are involved in the interactions with host cells, in particular via the CD81 receptor. The NS2 protein has a metalloprotease function involved in particular in NS2/NS3 cleavage. NS3 (631 amino acids, the first amino acid of the NS3 protein corresponds to that located at position 1007 in the sequence of the HCV polyprotein) and NS4 participate in both a serine protease activity and an RNA helicase activity. The NS5A and NS5B proteins are RNA polymerases that participate in replication of the HCV genome. In the following text, the amino acid numbering is indicated with reference to the sequence of the polyprotein of HCV genotype 1a (SWISSPROT P26664 and Choo et al., P.N.A.S., 1991, 88, 2451-2455); it should be noted that this numbering is identical for all the HCV genotypes insofar as their polyproteins are the same size.

HCV can be classified into 6 genotypes or clades (denoted from 1-6) and up to 100 subtypes (denoted a, b, c, etc.). This classification, according to Simmonds et al. (J. Gen. Virol., 1993, 74, 2391-), is based on a phylogenetic analysis of the NS5 sequences. The inter-genotypic variability also affects, but to varying degrees, the other viral proteins. Genotypes 1-3 have a worldwide distribution, genotypes 4 and 5 are present mainly in Africa, while genotype 6 is mainly distributed in Asia. Within an infected individual, the HCVs do not represent a homogeneous species, but constitute numerous quasispecies. This phenomenon is the result of the appearance of mutations in the genome due to the low replication fidelity of the viral polymerase.

HCV causes acute infections in humans which may be naturally resolved, but can also result in persistence of the virus, in more than 80% of cases. The acute phase is relatively benign, with only 20 to 30% of infected individuals developing clinical symptoms or signs. Approximately 60% of patients experience an increase in transaminases, reflecting a chronic hepatitis. Epidemiological studies indicate that 20% of individuals carrying the virus develop cirrhosis after about 20 years, followed by hepatic decompensation, or even a hepatocarcinoma. In industrialized countries, HCV is responsible, in total, for 20% of acute hepatitis forms, for 60% of hepatocellular carcinomas and for 30% of liver transplants. Current treatments (interferon-alpha/ribavirin) make it possible to cure approximately 50% of patients in the chronic phase.

Unlike HIV infection, HCV infection is not systematically persistent. Many infected patients in fact manage to overcome the infection through an effective immune response. T helper lymphocytes could be involved in the early control of the viral replication (Thimme et al.; J. Exp. Med., 2001, 194, 1395). In fact, a high activity of T helper lymphocytes specific for the C, NS3, NS4 and NS5 proteins, and of the Th1 type (i.e. secreting interleukins such as IL2 and IFNγ), is associated with the spontaneous recovery process (Diepolder et al., Lancet, 1995, 346, 1006-); Pape et al., J. Virol. Hepat. Suppl., 1999, 1, 36; Gerlach et al., Gastroenterology, 1999, 117, 9333. Conversely, a Th2 T helper response (i.e. characterized by the secretion of IL4 and IL5) is associated with a poor prognosis for the evolution of the HCV infection (Tai et al., J. Biomed. Sci., 2001, 8, 321). The involvement of T helper lymphocytes in the evolution of HCV infection has been supported by immunogenetic analyses revealing a correlation between spontaneous recovery and HLA class II alleles (HLA-DRB1*1101/DQB1*03; Thurzs et al., J. Virol. Hepat., 1997, 4, 215).

On the other hand, in chronic infected patients, a restricted number of T epitopes are capable of restimulating, in vitro, T lymphocytes derived from peripheral blood or from liver samples, and it is therefore difficult to measure the production of y interferon by T helper lymphocytes after stimulation with viral proteins (core, NS3, NS4 and NS5). It would seem that the orientation of the immune response toward a Th0/Th2-type profile, and also a tolerance to the viral antigens, are the cause of this loss of antiviral immunity and therefore of the persistence of HCV.

Cell responses mediated by CD4⁺ T helper lymphocytes are very often involved in antiviral mechanisms. CD4⁺ T lymphocytes, which are antigen-specific, are in fact capable of detecting the presence of a pathogenic agent and, under the effect of this recognition, of triggering an immune response. The antigen recognition in fact results in their activation. Once activated, the CD4+ T lymphocytes secrete most of the cytokines necessary for the recruitment of effector cells, namely cytotoxic CD8+ lymphocytes and antibody-producing B lymphocytes. They are also involved in the activation of cells via cell contacts and, for example, induce the activation, via CD40, of antigen-presenting dendritic cells. Finally, they can themselves play the role of effectors by producing antiviral lymphokines such as IFN-γ and TNF-α.

Whether the beneficial role of CD4+ T lymphocytes in resolving HCV infection is the result of a direct or indirect action, it constitutes an important argument in favor of vaccines directed against HCV and capable of stimulating a strong Th1-type CD4+ response. In particular, the use of peptides capable of stimulating these cells would seem to constitute a very advantageous vaccine approach that is both effective and economically viable. However, the peptides recognized by T helper lymphocytes are difficult to define due to the polymorphism of the HLA II molecules.

In fact, CD4+ T lymphocytes only recognize antigens in the form of peptides presented by HLA II molecules. More precisely, the activation of CD4+ T lymphocytes takes place under the effect of the presentation of peptides by the HLA II molecules carried by antigen-presenting cells (APCs). These peptides, called T epitopes, are the result of proteolytic degradation of the antigens by the APC. They are variable in length, generally from 13 to 25 amino acids, and have a sequence which makes them capable of binding to the HLA II molecules. The HLA II molecules are heterodimers, capable of binding a considerable repertoire of peptides having very different sequences. Four types of HLA II molecules exist per individual (2 HLA-DR, 1 HLA-DQ and 1 HLA-DP), the HLA-DR molecule, the β-chain of which is encoded by the DRB1 gene, being the most expressed. These isoforms have binding properties that are different from one another, which implies that they can bind different peptides of the same antigen. They are very polymorphic and at least 200 different alleles have been counted for DRB1. The molecules derived from the DRB3, DRB4, DRB5 and DPB1 loci are less polymorphic. Because of this polymorphism, each individual recognizes, in an antigen, a set of peptides the nature of which depends on the HLA II molecules that characterize it.

To the polymorphism of the HLA II molecules must be added the difficulty in finding sequences that are conserved between the various strains of HCV.

One of the means most commonly used for defining CD4+ T helper epitopes is to measure the ability of peptides to cause the mononuclear cells of individuals who have been in contact with the antigen under consideration to proliferate.

A certain number of authors have identified HCV peptides as being T epitopes in the patients studied:

Diepolder et al., mentioned above, have shown the existence of HCV epitopes derived from NS3 protein (NS3 1248-1261, NS3 1388-1407, NS3 1450-1469) that are recognized by T helper lines obtained from patients in the acute phase.

Lamonaca et al. (Hepatology, 1999, 30, 10888) have identified five major epitopes (core 21-40, NS3 1253-1272, NS4 1767-1786, NS4 1907-1926, NS4 1909-1929), according to a similar approach. These peptides are at the same time immunodominant, conserved, and presented by several HLA II molecules; 4 immunodominant peptides are more particularly described: C 21-40, NS3 1253-1272 and NS4 1767-1786, 1907-1926 and 1909-1929. The assays for binding to HLA II molecules that are most common in the Caucasian and worldwide population (DR1, DR4, DR7, DR11, DR15, DRB5, DR6, DR8 and DR9) given in table 3 of that document show that only the peptides derived from NS3 and from NS4 (NS3 1253-1272, NS4 1767-1786 and NS4 1909-1929) bind with a binding activity of less than 1000 nM to at least 4 of the HLA II molecules whose frequency is greater than 5% in the Caucasian population (DR1, DR4, DR11, DR7, DR15 and DRB5). On the other hand, the peptide derived from the C protein (C 21-40) binds with high affinity only to one of these HLA II molecules, namely DR15.

Tabatabai et al. (Hum. Immunol. 1999, 60, 105) have identified several major epitopes (NS3 1384-1401, NS3 1454-1471) in a single chronically infected patient; these epitopes are capable of inducing proliferation and the production of IL-2.

Godkin et al. (Eur. J. Immunol., 2001, 31, 1438; see also PCT International Application PCT WO 02/34770 in the name of Imperial College Innovations Ltd) have identified several T help epitopes restricted by the HLA-DR11 allele, in several non-viremic or chronically infected patients; these are DR11-restricted peptides and polypeptides and not mixtures of peptides that bind to the HLA II molecules most common in the Caucasian population.

Hoffmann et al. (Hepatology, 1995, 21, 632) have demonstrated that the peripheral blood mononuclear cells of patients in the chronic phase recognize several peptides derived from the core protein.

Other similar studies exist, all based on the responses of mononuclear cells from patients infected with HCV (Woitas et al., J. Immunol., 1997, 159, 1012; Lechmann et al., Hepatology, 1996, 24, 790; Leroux-Roels et al., Hepatology, 1996, 23, 8; Lohr et al., Liver, 1996, 16, 174).

The T epitope peptides identified in these studies, which are derived from the most conserved HCV proteins (C and NS3), are given in table 1.

TABLE I
Sequences of the C and NS3 peptides identified
as being T epitopes by means of proliferation assays

| | HLA | | | HLA | |
|---|---|---|---|---|---|
| Peptide | restriction | Reference | Peptide | restriction | Reference |
| C1-24 | — | Woitas | C131-150 | — | Hoffmann |
| C21-40 | DRB1*1101 | Lamonaca | C133-152 | — | Leroux-Roels |
| | DQB1*0301 | | | | |
| C20-44 | | Woitas | C138-162 | | Lechmann |
| | | Lechmann. | | | |
| C23-42 | — | Hoffmann | C141-155 | DR11 | Godkin |
| | | | | | WO 02/34770 |
| C31-45 | DR11 | Godkin | C142-161 | — | Hoffmann |
| | | WO 02/34770 | | | |
| C39-63 | DR4 | Lechmann | C141-160 | — | Woitas |
| C41-60 | — | Lamonaca | C145-164 | — | Leroux-Roels |
| C47-70 | — | Lohr | C148-172 | DR11 | Woitas |
| | | | | | Lechmannn |
| C55-74 | — | Hoffmann | C153-172 | — | Hoffmann |
| C66-85 | — | Hoffmann | C157-176 | — | Leroux-Roels |
| C73-92 | — | Leroux-Roels | C161-180 | DRB1*08032 | Kaneko |
| C71-90 | — | Lamonaca | C168-192 | — | Woitas |
| | | | | | Lechmann |
| C79-103 | — | Woitas | N1205-1221 | DR11 | Godkin |
| | | Lechmann | | | WO 02/34770 |
| C81-100 | — | Lamonaca | N1242-1261° | | Diepolder |
| C85-104 | — | Leroux-Roels | N1248-1261° | DRB1*0404 | Diepolder |
| | | | | DRB1*1101 | |
| | | | | DRB1*1201 | |
| | | | | DRB1*1301 | |
| | | | | DRB1*1601 | |
| C91-110 | — | Lamonaca | N1248-1267 | | Diepolder |
| C97-116 | — | Leroux-Roels | N1245-1269 | DR11 | Godkin |
| | | Lamonaca | | | |
| C101-115 | — | Lohr | N1253-1272 | DQB1*0301 | Lamonaca |
| C111-130 | DRB1*08032 | Kaneko | N1293-1331 | — | Tabatai |
| C109-128 | — | Leroux-Roels | N1388-1407 | DRB1*1501 | Diepolder |
| | | Hoffmann | | | |
| C120-139 | — | Hoffmann | N1384-1401 | — | Tabatai |
| C121-140 | — | Leroux-Roels | N1450-1469 | DRB1*1302 | Diepolder |
| C118-142 | | Woitas | N1447-1464 | — | Tabatai |
| C128-152 | — | Woitas | N1454-1471 | — | Tabatai |
| | | Lechmann | | | |

— Not determined/°and variants of these peptides including the minimum peptide N1251-1259.

It emerges from table I that the sequences of the T epitopes vary from one study to another, reflecting the lack of precision of this approach. It is in fact difficult, in view of the diversity of the responses observed, to define T epitope sequences for the entire Caucasian population. These sequences are only suitable for the patients who were used in these studies. These differences in response are explained, firstly, by the representativeness of the samples, which is not evaluated. In particular, in the studies where the patients are not typed for their HLA molecules, nobody knows whether the various alleles are represented according to the frequencies of the general population. The response of many patients to a particular peptide may then result from a sampling bias and not from the effective ability of a peptide to be recognized by all the patients. Secondly, in the case of HCV infection, several studies have been carried out on chronically infected patients. Because of the persistence of the virus in these individuals, it is possible that they exhibit a tolerance to the T epitopes that are the most effective in being presented. In this context, the most valuable epitopes could have disappeared from the immune response, which would be maintained for epitopes which are less stimulating but which do not manage to eliminate the virus. This hypothesis is, moreover, put forward by Tabatabai et al., mentioned above. These proliferation assays are therefore insufficient to define sequences suitable for immunization of the entire population.

Among the peptides having T lymphocyte-stimulating activity that have been identified, only some bind to HLA II molecules (table II).

TABLE II
Binding of the C and NS3 peptides to HLA II
molecules

| Peptides | Binding to |
|---|---|
| C21-40 | DRB1*1501 |
| N1242-1261 | DRB1*0101, 1501, 0401, 0404, |
| | 1101, 1302, 0701, 0802, 0901, |
| | DRB5* 0101 |
| N1248-1267 | DRB1*0101, 1501, 0401, 0404 1101, |
| | 1302, 0701, 0802, 0901 |
| N1248-1261 | DRB1*0101, 1501, 0401, 0404, |
| | 0405, 1101, 1302, 0802, 0901 |
| N1253-1272 | DRB1*0101, 0401, 1101, 701, 1501, |
| | 1302, 0802, 0901 |
| N1388-1407 | NONE |
| N1450-1469 | NONE |

In the article by Diepolder et al., mentioned above, the authors in fact studied, for 5 peptides of the NS3 protein, the ability to bind HLA II molecules commonly encountered. In fact, they showed that the NS3 1248-1261 epitope or variant peptides (1242-1261 and 1248-1267) exhibit a binding capacity for 9 or 10 HLA-DR alleles, namely: DRB1*0101, 1501, 0401, 0404, 0405, 1101, 1302, 0802, 0901 and DRB5*0101. On the other hand, peptides 1388-1407 and 1450-1469 are inactive.

In the article by Lamonaca et al., mentioned above, the authors also showed, by means of binding assays, that the core peptide 21-40 binds to DRB1*1501 and that the NS3 peptide 1253-1272 binds to DRB1*0101, 0401, 1101, 701, 1501, 1302, 0802 and 0901.

It emerges from the above that no HCV peptide that binds to the HLA II molecules most commonly encountered in the Caucasian population was identified for the C protein, which is the most conserved and therefore particularly suitable for immunization against the various genotypes of HCV. In addition, among the peptides of the NS3 protein having T lymphocyte-stimulating activity that were studied, a single peptide binds to the HLA II molecules most commonly encountered in the Caucasian population.

There is a large number of HLA II molecules whose distribution is not even in the world. Thus, in a given population, a set of alleles includes, on its own, most of the alleles of the population. For example, in France, which is a population characteristic of the Caucasian population (USA, Europe), only 7 alleles of the DRB1 locus exceed 5%. These are the alleles DRB1*0101, DRB1*0301, DRB1*0401, DRB1*0701, DRB1*1101, DRB1*1301 and DRB1*1501 which represent, by themselves, 64% of the population. These same alleles are the most abundant HLA-DR alleles in the other Caucasian populations. Their frequency ranges between 53% (in Spain) and 82% (in Denmark). For the United States and Canada, they represent respectively 58 and 55% of the alleles of the population. The HLA-DRB3, -DRB4 and -DRB5 molecules, which are HLA-DR molecules in which the β-chain is not encoded by the DRB1 gene, are also present with high allelic frequencies, since they are less polymorphic than the DRB1 molecules. Their allelic frequency is in fact 9.2% for DRB3*0101, 28.4% for DRB4*0101 and 7.9% for DRB5*0101. They therefore cover, by themselves, 45% of the allelic frequency. Finally, the HLA-DP4 molecules, which include the molecules encoded by the DPB1*0401 and DPB1*0402 alleles, are the HLA II molecules that are the most abundant in Europe and in the United States. Their allelic frequency is in fact 40% and 11%, respectively, which means that one or other of them is found in approximately 76% of individuals. The peptides present in a peptide sequence and which bind all these alleles therefore include the T epitopes of the majority of the population.

For this reason, the inventors gave themselves the aim of providing for a set of peptides capable of being incorporated into an immunogenic composition and of stimulating anti-HCV CD4+ T lymphocytes in the majority of Caucasian individuals from Europe or from North America, so as to effectively induce a proliferative and multiepitope response (which makes use of several epitopes) specific for the components of the virus.

Such a set has the property of being effective in a large number of individuals, whereas the peptides of the prior art are active in a few individuals and are inactive in the majority of other individuals, because the latter do not recognize the HCV proteins by the same determinants.

To do this, the inventors have identified the sequences of peptides derived from the C and NS3 proteins of HCV that are restricted to the HLA II molecules predominant in Caucasian populations, and they have shown that the peptides derived from the C protein, preferably associated with peptides derived from the NS3 protein, effectively induce an immunogenic and protective response in a large number of individuals, which involves several epitopes.

In addition, these peptides which recognize HCV-specific T lymphocytes in infected patients are useful for diagnosing the immune state of these patients with respect to the hepatitis C virus.

Consequently, a subject of the present invention is a peptide mixture, characterized in that it includes at least two different peptides derived from the hepatitis C virus (HCV), at least one of which is a peptide derived from the C protein, that bind to at least four different HLA II molecules whose allelic frequency is greater than 5% in the Caucasian population, with a binding activity <1000 nM.

In accordance with the invention, said mixture also comprises, besides said C peptide as defined above, one or more peptides or lipopeptides containing one or more CD8+, CD4+ or B epitopes, and more particularly epitopes derived from an HCV protein, in particular at least one peptide derived from the NS3 protein, that binds to at least four different HLA II molecules whose allelic frequency is greater than 5% in the Caucasian population, with a binding activity <1000 nM.

The invention encompasses the peptides derived from the C and NS3 proteins of any genotype of HCV.

In accordance with the invention, the term “different HLA II molecules” is intended to mean both HLA DP and DQ molecules encoded by different alleles and DR molecules encoded by different genes or different alleles of the same gene.

Advantageously, said HLA II molecules are chosen from the molecules HLA-DR1, HLA-DR3, HLA-DR4, HLA-DR7, HLA-DR11, HLA-DR13, HLA-DR15, HLA-DRB3, HLA-DRB4, HLA-DRB5 and HLA-DP4.

Particularly advantageously, said HLA II molecules are encoded, respectively, by the HLA alleles DRB1*0101, DRB1*0301, DRB1*0401, DRB1*0701, DRB1*1101, DRB1*1301, DRB1*1501, DRB3*0101, DRB4*0101, DRB5*0101, DP*0401 and DP*0402.

Such a peptide mixture makes it possible to obtain, surprisingly, a CD4+ T proliferative response (stimulation of CD4+ T lymphocytes) in the vast majority of the Caucasian population to be protected and whatever the HCV genotype concerned; it may thus be considered that such a mixture constitutes a first step toward a “universal” immunogenic composition capable of being effectively used in a vaccine.

According to an advantageous arrangement of said mixture, the peptides derived from the C protein of the hepatitis C virus are selected from the group consisting of:

a) the peptides corresponding to positions 19-47, 27-51, 31-57, 104-133 and 127-167,

b) the peptides of at least 11 amino acids included in the peptides as defined in a), and

c) the peptides derived from the peptides as defined in a) or in b) by substitution, with alanine residues (C→A), of cysteine residue(s) at position +1 or +2, relative to the amino acid residue at the N-terminal position and/or at position −1, −2 or −3, relative to the amino acid residue at the C-terminal position.

Advantageously, the peptides of at least 11 amino acids as defined in b) are selected from the group consisting of:

the peptide included in peptide 27-51 that corresponds to positions 27-41,

the peptide included in peptide 31-57 that corresponds to positions 31-45, and

the peptides included in peptide 127-167 that correspond, respectively, to positions 127-149, 131-145, 131-148, 131-167, 134-148 and 148-167.

Advantageously, the peptides as defined in c) are selected from the group consisting of the peptide derived from the C peptide 127-149 of sequence TAGFADLMGYIPLVGAPLGGAAR (SEQ ID NO:5).

According to another advantageous arrangement of said mixture, the peptides derived from the NS3 protein are selected from the group consisting of:

d) the peptides corresponding, respectively, to positions 1007-1037, 1036-1055, 1052-1072, 1076-1093, 1127-1153, 1149-1172, 1174-1195, 1190-1212, 1206-1239, 1246-1275, 1275-1304, 1361-1387, 1377-1403, 1404-1432, 1456-1481, 1495-1513, 1524-1553 and 1552-1583,

e) the peptides of at least 11 amino acids included in the above peptides, and

f) the peptides derived from the peptides as defined in d) or in e) by substitution, with alanine residues (C→A), of cysteine residue(s) at position +1 or +2, relative to the amino acid residue at the N-terminal position and/or at position −1, −2 or −3, relative to the amino acid residue at the C-terminal position.

Advantageously, the peptides of at least 11 amino acids as defined in e) are selected from the group consisting of:

the peptides included in peptide 1007-1037 that correspond, respectively, to positions 1007-1021, 1015-1029, 1015-1037, 1019-1033 and 1020-1034,

the peptides included in peptide 1174-1195 that correspond, respectively, to positions 1174-1188, 1174-1192 and 1178-1192,

the peptides included in peptide 1190-1212 that correspond, respectively, to positions 1190-1204 and 1192-1206,

the peptides included in peptide 1246-1275 that correspond, respectively, to positions 1246-1260, 1246-1264, 1250-1264 and 1261-1275,

the peptides included in peptide 1377-1403 that correspond, respectively, to positions 1381-1395, 1381-1397, 1381-1403 and 1383-1397,

the peptide included in peptide 1495-1513 that corresponds, respectively, to positions 1495-1509,

the peptides included in peptide 1524-1553 that correspond, respectively, to positions 1524-1552, 1524-1538, 1528-1542, 1528-1552, 1529-1543, 1534-1548, 1538-1552 and 1540-1553, and

the peptides included in peptide 1552-1583 that correspond, respectively, to positions 1559-1573 and 1563-1577.

Advantageously, the peptides as defined in f) are selected from the group consisting of:

the peptide derived from peptide 1076-1093 of sequence GVAWTVYHGAGTRTIASP (SEQ ID NO:10),

the peptide derived from peptide 1149-1172 of sequence RGSLLSPRPISYLKGSSGGPLLAP (SEQ ID NO:13),

the peptide derived from peptide 1377-1403 of sequence GKAIPLEVIKGGRHLIFCHSKKKADEL (SEQ ID NO:20),

the peptide derived from peptide 1456-1481 of sequence TAVTQTVDFSLDPTFTIETITLPQDA (SEQ ID NO:22), and

the peptide derived from peptide 1524-1553 of sequence GAAWYELTPAETTVRLRAYMNTPGLPVAQD (SEQ ID NO:24) and the peptides included in the sequence SEQ ID NO:24 that correspond, respectively, to positions 1524-1538, 1524-1552, 1528-1552, 1538-1552 and 1540-1553.

According to another advantageous embodiment of said mixture, it includes peptides derived from the C and NS3 proteins of HCV genotype 1, preferably subtype 1a or 1b.

According to yet another advantageous embodiment of said mixture, it includes 2 to 6 different peptides derived from the C and NS3 proteins, as defined above, all the peptides binding to at least 10 HLA II molecules whose allelic frequency is greater than 5% in the Caucasian population.

According to an advantageous arrangement of this embodiment, said peptides are selected from the group consisting of the peptides derived from the C protein that correspond, respectively, to positions 27-51, 131-167, 127-149, 131-148 and 148-167 and the peptides derived from the NS3 protein that correspond, respectively, to positions 1007-1037, 1015-1037, 1036-1055, 1174-1192, 1190-1212, 1246-1264, 1381-1403, 1381-1397, 1524-1553, 1528-1552 and 1552-1583.

For example:

the C peptide 19-47 binds with good affinity to the DR1, DR7, DR11, DR13, DR15, DRB5 and DP402 molecules encoded by the alleles as defined above,

the C peptide 31-57 binds with good affinity to the DR1, DR7, DR1, DR13, DR15, DRB5, DP401 and DP402 molecules encoded by the alleles as defined above,

the C peptide 104-133 binds with good affinity to the DR1, DR7, DR11 and DRB5 molecules encoded by the alleles as defined above,

the C peptide 127-149 binds with good affinity to the DR1, DR7, DR1, DR15 and DRB5 molecules encoded by the alleles as defined above,

the NS3 peptide 1007-1037 binds with good affinity to the DR1, DR3, DR4, DR7, DR1, DR13, DR15, DRB4, DRB5 and DP402 molecules encoded by the alleles as defined above,

the NS3 peptide 1036-1055 binds with good affinity to the DR1, DR4, DR11, DRB4 and DRB5 molecules encoded by the alleles as defined above,

the NS3 peptide 1052-1080 binds with good affinity to the DR1, DR4, DR7, DR11, DR15, DRB4 and DRB5 molecules encoded by the alleles as defined above,

the NS3 peptide 1076-1093 binds with good affinity to the DR1, DR7, DR11, DR15 and DRB5 molecules encoded by the alleles as defined above,

the NS3 peptide 1127-1153 binds with good affinity to the DR1, DR7, DR11, DR13 and DRB5 molecules encoded by the alleles as defined above,

the NS3 peptide 1149-1172 binds with good affinity to the DR1, DR7, DR15, DRB4 and DRB5 molecules encoded by the alleles as defined above,

the NS3 peptide 1174-1195 binds with good affinity to the DR1, DR4, DR7, DR11, DR15, DRB4 and DRB5 molecules encoded by the alleles as defined above,

the NS3 peptide 1190-1212 binds with good affinity to the DR1, DR4, DR7, DR1, DR15 and DRB5 molecules encoded by the alleles as defined above,

the NS3 peptide 1206-1239 binds with good affinity to the DR1, DR4, DR7, DR11 and DRB5 molecules encoded by the alleles as defined above,

the NS3 peptide 1246-1275 binds with good affinity to the DR1, DR4, DR7, DR1, DR13 and DR15 molecules encoded by the alleles as defined above,

the NS3 peptide 1275-1304 binds with good affinity to the DR1, DR4, DR7, DR11, DR15, DRB3 and DP401 molecules encoded by the alleles as defined above,

the NS3 peptide 1361-1387 binds with good affinity to the DR1, DR7, DR15 and DRB5 molecules encoded by the alleles as defined above,

the NS3 peptide 1377-1403 binds with good affinity to the DR1, DR7, DR11, DR13, DRB4 and DRB5 molecules encoded by the alleles as defined above,

the NS3 peptide 1404-1432 binds with good affinity to the DR1, DR4, DR7, DR15 and DRB5 molecules encoded by the alleles as defined above,

the NS3 peptide 1456-1481 binds with good affinity to the DR1, DR3, DR4, DR7, DR1, DR13, DR15, DRB3, DRB4 and DRB5 molecules encoded by the alleles as defined above,

the NS3 peptide 1495-1513 binds with good affinity to the DR1, DR7, DR1, DR15, DRB3, DRB4 and DRB5 molecules encoded by the alleles as defined above,

the NS3 peptide 1524-1553 binds with good affinity to the DR1, DR4, DR7, DR1, DR15, DRB3, DRB4, DRB5 and DP402 molecules encoded by the alleles as defined above,

the NS3 peptide 1552-1538 binds with good affinity to the DR1, DR7, DR1, DR15, DRB5, DP401 and DP402 molecules encoded by the alleles as defined above.

In accordance with the invention, the peptides included in said mixture are in the form either of individualized peptides or of a fusion protein comprising a sequence of the peptides of said mixture, with the exclusion of the sequence corresponding to the fusion of the peptides C 31-45, C 141-155 and NS3 1207-1221.

Said individualized peptides are prepared according to the conventional methods for solid-phase parallel synthesis and said fusion protein is prepared according to the conventional recombinant DNA techniques, in a suitable expression system.

Said fusion protein comprises the sequences of said peptides directly linked to one another via a peptide bond or else separated by exogenous sequences, i.e. sequences other than those present at this position in the sequence of the C and NS3 proteins of HCV, in particular the sequences of other CD4+ or CD8+ T epitopes, or B epitopes, for example of HCV.

A subject of the present invention is also a nucleic acid molecule, characterized in that it encodes a fusion protein as defined above.

A subject of the invention is also any recombinant vector, in particular plasmid or virus, comprising at least one nucleic acid molecule as defined above, placed under the control of the elements required for transcription of said molecule, in particular under the control of a promoter and of a transcription terminator.

The invention also relates to the host cells, in particular bacteria, yeast or mammalian cells, transformed using a vector as defined above, so as to stably integrate into their genome or to stably maintain at least one nucleic acid molecule as defined above.

A subject of the present invention is also an anti-HCV immunogenic composition, characterized in that it comprises at least:

one mixture of peptides derived from a C protein and from an NS3 protein of HCV, as defined above, and/or

one nucleic acid molecule as defined above, or

one suitable vector as defined above, in particular a virus, in combination with at least one pharmaceutically acceptable vehicle and, optionally, at least one adjuvant.

The adjuvants used are adjuvants conventionally used in vaccine compositions, such as alumina hydroxide and squalene.

Use may be made, inter alia, of viral vectors such as adenoviruses, retroviruses, lentiviruses and AAVs, into which the sequence of interest has been inserted beforehand; it is also possible to associate said sequence (isolated or inserted into a plasmid vector) with a substance capable of providing protection for said sequences in the organism or allowing it to cross the host-cell membrane, for example a preparation of liposomes, of lipids or of cationic polymers, or alternatively to inject it directly into the host cell, in the form of naked DNA.

For example, the use of naked DNA for immunization constitutes an effective vaccine approach: it consists in injecting into the host organism to be immunized a naked DNA encoding a protein antigen; this DNA allows prolonged synthesis of the antigen by the host cells and also long-lasting presentation of this antigen to the immune system.

According to an advantageous embodiment of said immunogenic composition, said peptides are in the form of modified peptides or else peptides associated with liposomes or with lipids, in particular in the form of lipopeptides.

The lipid component of the lipopeptide is in particular obtained by addition of a lipid unit on an α-amino function of said peptides or on a reactive function of the side chain of an amino acid of the peptide component; it may comprise one or more C₄₋₂₀ fatty acid-derived chains, optionally branched or unsaturated (palmitic acid, oleic acid, linoleic acid, linolenic acid, 2-aminohexadecanoic acid, pimelautide, trimexautide) or a derivative of a steroid. The method for preparing such lipopeptides is in particular described in international applications WO 99/40113 or WO 99/51630. The preferred lipid component is in particular represented by an N^{α}-acetyl-lysine N^{ε} (palmitoyl) group, also called Ac-K(Pam).

The modified peptide is in particular obtained by a modification of at least one peptide bond —CO—NH— of the peptide chain of said peptides by the introduction of a retro or retro-inverso (—NH—CO—) type bond or of a bond different from the peptide bond (methyleneamino, carba, ketomethylene, methyleneoxy, etc.) or else by substitution of at least one amino acid of the peptide chain of said peptides with a non-proteinogenic amino acid, i.e. an amino acid that is not part of the constitution of a natural protein, in particular an amino acid in which the carbon carrying the side chain, i.e. the —CHR— group, is replaced with a unit that is not part of the constitution of a natural amino acid.

According to another advantageous embodiment of said immunogenic composition, said peptide mixture is combined:

with one or more peptides or lipopeptides containing one or more CD8+ epitopes (recognized specifically by cytotoxic T lymphocytes and presented by HLA I molecules) and more particularly CD8+ epitopes derived from an HCV protein, such as the C peptides 2-10, 28-36, 35-44, 41-49, 42-50, 85-98, 88-97, 127-140, 131-140, 132-140, 167-176, 178-187, 181-190; the E1 peptides 220-227, 233-242, 234-242, 363-371; the E2 peptides 401-411, 460-469, 489-496, 569-578, 621-628, 725-733; the NS2 peptides 826-838, 838-845; the NS3 peptides 1073-1081, 1169-1177, 1287-1296, 1395-1403, 1406-1415; the NS4A peptides 1585-1593, 1666-1675; the NS4B peptides 1769-1777, 1789-1797, 1807-1816, 1851-1859; the NS5A peptide 2252-2260 and the NS5B peptides 2588-2596 and 2727-2735 (Rehermann et al., Current Topics in Microbiology and Immunology, 2000, 242; 299),

with other peptides comprising multiple CD4+ epitopes, such as the tetanus toxin TT peptide (positions 830-846), the Influenza hemagglutinin HA peptide (positions 307-319), PADRE (Pan DR Epitope, Alexandre J. et al., Immunity, 1994, 1, 9, 751-761) and the Plasmodium falciparum LSA3 peptide, and/or

with one or more peptides or lipopeptides containing one or more B epitopes, more particularly B epitopes derived from an HCV protein that are specifically recognized by antibodies directed against these epitopes, such as the C peptide 5-27 (Khanna et al., Acta Virologica, 1998, 42, 141-145), the NS4 peptide 1698-1719 (Khanna et al., mentioned above) and the NS5 peptide 2295-2315 (Khudyakov et al., Virology, 1995, 206, 666-672).

The C and NS3 peptides according to the invention, included in the mixtures, as defined above were advantageously selected using an HLA II/peptide binding assay comprising:

purifying the HLA II molecules of interest, i.e. those relating to more than 5% of a given population and in particular the HLA molecules DR1, DR3, DR4, DR7, DR11, DR13, DR15, DRB3, DRB4, DRB5 and DP4,

incubating the HLA II molecules thus purified, with various concentrations of overlapping fragments covering the sequence of the C protein or of the NS3 protein and with a reagent R1 or a tracer consisting of a peptide fragment combined with a nonradioactive label, such as biotin, and the sequence of which is different from said peptides; the reagent R1 or tracer is chosen in such a way that it has affinity with respect to one of the HLA II molecules of interest, such that it can be used at a concentration <200 nM,

transferring the complexes obtained onto an ELISA plate, precoated with an antibody specific for all DR or DP molecules,

visualizing the HLA II molecules/reagent R1 complexes attached to the bottom of the plate by means of suitable conjugates, such as streptavidin-phosphatase, and of a fluorescent substrate,

selecting the peptides comprising different epitopes, i.e. the most representative of the various zones of interaction between the C protein or the NS3 protein and the HLA II molecules, and

choosing the most suitable peptides according to the frequency of the alleles with respect to which they exhibit a binding activity <1000 nM, preferably <800 nM, corresponding to the concentration of these peptides which inhibits 50% of the binding of the reagent R1 (IC₅₀).

These assays make it possible, unambiguously, to associate with each HLA II molecule the sequences of the fragments capable of binding thereto or, on the contrary, which do not bind thereto.

This approach makes it possible to define immunogenic compositions including peptides which bind to the greatest number of different HLA II molecules and which can thus be advantageously protective for the majority of patients, even if their HLA genotypes are not known.

This approach also has the advantage of allowing the selection of peptides that are significantly more specific with respect to HCV than in the approaches that seek to select peptides on the basis of their ability to stimulate CD4+ T lymphocytes (proliferation assays).

The incubation conditions are specific to each HLA II molecule (incubation time, pH, reagent R1, concentration of HLA II or of reagent R1).

The reagent R1 is selected from the group consisting of the following sequences:
PKYVKQNTLKLAT (HA 306-318 SEQ ID NO: 75), specific for the alleles DRB1*0101, DRB1*0401, DRB1*1101 and DRB5*0101,
EAEQLRAYLDGTGVE (A3 152-166, SEQ ID NO: 79), specific for the allele DRB1*1501,
AKTIAYDEEARGLE (MT 2-16, SEQ ID NO: 77), specific for the allele DRB1*0301,
AAYAAAKAAALAA (YKL, SEQ ID NO: 76), specific for the allele DRB1*0701,
TERVRLVTRHIYNREE (B1 21-36, SEQ ID NO: 78), specific for the allele DRB1*1301,
ESWGAVWRIDTPDKLTGPFT (LOL 191-210, SEQ ID NO: 80), specific for the allele DRB3*0101,
AGDLLAIETDKATI (E2/E168, SEQ ID NO: 81), specific for the allele DRB4*0101, and
EKKYFAATQFEPLAARL (Oxy 271-287, SEQ ID NO: 82), specific for the alleles DP*0401 and DP*0402.

Other reagents R1 can be used, in particular those described in Southwood et al. (J. Immunol., 1998, 160, 3363-3373).

A subject of the present invention is also a vaccine intended for the prevention and treatment of HCV infections, characterized in that it includes an immunogenic composition as defined above.

A subject of the present invention is also peptides derived from the C protein or from the NS3 protein of an HCV, in particular genotype 1a or 1b, characterized in that they are selected from the group consisting of: the peptides derived from the C protein, as defined above, with the exclusion of the peptide C 31-45, C 21-40, C 20-44, C 23-42, C 111-130, C 109-128, C 128-152, C 131-150, C 133-152, C 138-162, C 141-155, C 142-161, C 141-160 and C 145-164, and the peptides derived from the NS3 protein, chosen from:

the peptides corresponding, respectively, to positions 1007-1037, 1036-1055, 1052-1072, 1076-1093, 1127-1153, 1149-1172, 1174-1195, 1190-1212, 1206-1239, 1275-1304, 1361-1387, 1377-1403, 1404-1432, 1456-1481, 1495-1513, 1524-1553 and 1552-1583 and the peptides of at least 11 amino acids included in the above peptides, with the exclusion of the peptides NS3 1384-1401 and NS3 1207-1221,

the peptides corresponding, respectively, to positions 1246-1260 and 1261-1275,

the peptides derived from the above peptides by substitution, with aniline residues (C→A), of cysteine residue(s) located at position +1 or +2, relative to the amino acid residue at the N-terminal position and/or at position −1, −2 or −3, relative to the amino acid residue at the C-terminal position, and

the peptides derived from the above peptides, as defined above.

Such peptides which contain a CD4+ epitope, capable of having a binding activity <1000 nM, preferably <800 nM, with respect to at least four different HLA II molecules as defined above, are capable of being recognized by CD4+ T lymphocytes specific for said peptides that are present in patients infected with HCV and are therefore useful as reagents for diagnosing an HCV infection.

A subject of the present invention is also a diagnostic reagent, characterized in that it comprises at least one of the C or NS3 peptides as defined above, said peptides being optionally labeled or complexed, in the form of multimeric complexes.

A subject of the present invention is also a method for evaluating the immune state of an individual, characterized in that it comprises a step consisting in detecting the presence of CD4+ T cells specific for the C and/or NS3 peptides as defined above; said detection is advantageously carried out by means of one of the following assays: proliferation assay, ELISPOT assay [see, for example, international application WO 99/51630 or Gahéry-Ségard et al. J. Virol., 2000, 74, 1964-)] or flow cytometry in the presence of multimeric complexes made of up said E6 and/or E7 peptides.

More precisely:

* as regards the proliferation assay:

A suspension of cells (PBMCs, CD8+ cell-depleted PBMCs, T lymphocytes enriched beforehand by means of an in vitro culture step with the peptides selected according to the invention, or cloned T lymphocytes) is cultured for 3 to 5 days in the presence of the selected peptides and, as required, of suitable presenting cells such as dendritic cells, autologous or heterologous PBMCs, lymphoblastoid cells such as those obtained after infection with the EBV virus, or genetically modified cells. The cell proliferation is measured by incorporation of tritiated thymidine into the DNA of the cells. The peptides selected in accordance with the invention make it possible to reveal, in the initial suspension, the presence of cells specific for these peptides.

* as regards the ELISPOT assay:

The ELISPOT assay makes it possible to reveal the presence of T cells specific for a peptide selected in accordance with the invention and secreting IFN-γ.

More precisely, the T cells are revealed by measuring the secretion of IFN-γ after incubation of PBMCs from patients with the peptides selected according to the invention, in accordance with the method described in Gahéry-Ségard et al., J. Virol., 2000, 74, 1964.

* as regards the use of multimeric complexes and in particular of tetrameric complexes:

a biological sample, preferably peripheral blood mononuclear cells (PBMCs), is brought into contact with labeled tetrameric complexes produced from complexes of C and/or NS3 peptides as defined above with soluble HLA class II molecules, and

the labeled cells are analyzed, in particular by flow cytometry.

Advantageously, prior to bringing the biological sample into contact with said complex, it is enriched in CD4+ T cells, by bringing it into contact with anti-CD4 antibodies in order to enrich said sample.

The tetramers are prepared as specified, for example, in E. J. Novak et al. (J. Clin. Investig., 1999, 104, R63-R67) or in M. J. Kuroda et al. (J. Virol., 2000, 74, 18, 8751-8756).

Briefly, the tetramers are produced by incubating, for 72 hours at 37° C. and in a 10 mM citrate phosphate buffer containing 0.15 M NaCl at a pH of between 4.5 and 7, soluble, biotinylated HLA II molecules with a 10-fold excess of C and/or NS3 peptides identified and selected in accordance with the invention.

The tetramerized form is obtained by adding, to the preparation, streptavidin labeled with a fluorochrome in an amount that is four times less (mole for mole) than HLA II molecules. The entire mixture is incubated overnight at ambient temperature.

In order to use these tetramers, a suspension of cells (PBMCs, CD8+ cell-depleted PBMCs, T lymphocytes enriched beforehand by means of an in vitro culture step with the C and/or NS3 peptides selected in accordance with the present invention, or cloned T lymphocytes) is brought into contact with one or more tetramers (10 to 20 mg/ml) for 1 to 3 hours. After washing, the suspension is analyzed by flow cytometry: the labeling of the cells with the tetramers is visualized by virtue of the fact that these constructs are fluorescent.

Flow cytometry makes it possible to separate the cells labeled with the tetramers from the unlabeled cells and to thus effect a cell sorting.

A subject of the present invention is thus also a method for sorting HCV-specific T lymphocytes, characterized in that it comprises at least the following steps:

incubating a cell suspension to be sorted, or bringing it into contact, with one or more labeled tetramers formed from complexes of C and/or NS3 peptides as defined above with soluble HLA II molecules, and

sorting the cells labeled with the tetramers.

Besides the above provisions, the invention also comprises other provisions which will emerge from the following description, which refers to examples of implementation of the method which is the subject of the present invention and also to the attached drawings in which:

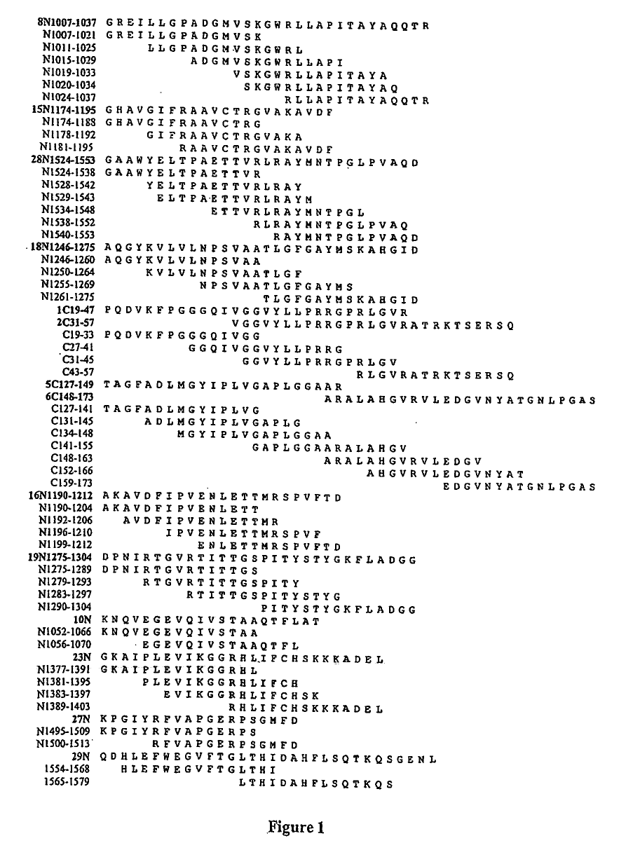

FIG. 1 illustrates the sequences of the peptides derived from the C and NS3 proteins which were studied. To simplify matters, the various peptides have been called 1C to 6C and 8N to 29N;

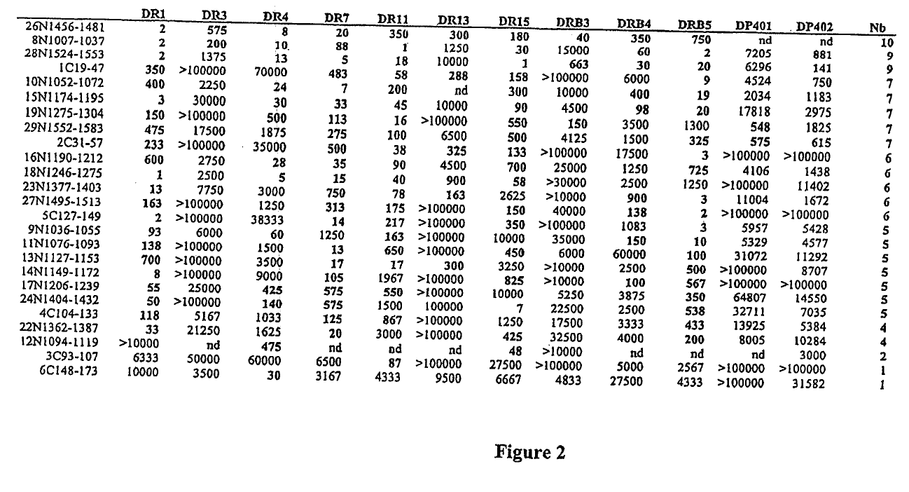

FIG. 2 illustrates the binding activity of a first series of peptides of the C and NS3 proteins, with respect to the HLA II molecules that are predominant in the Caucasian population. The values correspond to the IC₅₀ values, expressed in nM. The values less than 1000 nM, corresponding to the peptides exhibiting good affinity for the HLA II molecules, are indicated in bold. nd: not determined;

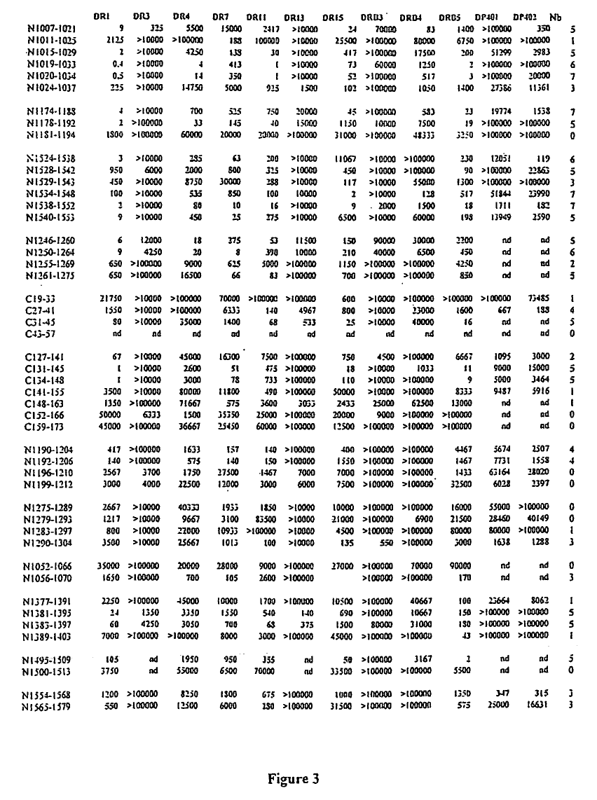

FIG. 3 illustrates the binding activity, with respect to the HLA II molecules that are predominant in the Caucasian population, of a second series of peptides of the C and NS3 proteins, derived from the first series. The values correspond to the IC₅₀ values, expressed in nM. The values less than 1000 nM, corresponding to the peptides exhibiting good affinity for the HLA II molecules, are indicated in bold. nd: not determined;

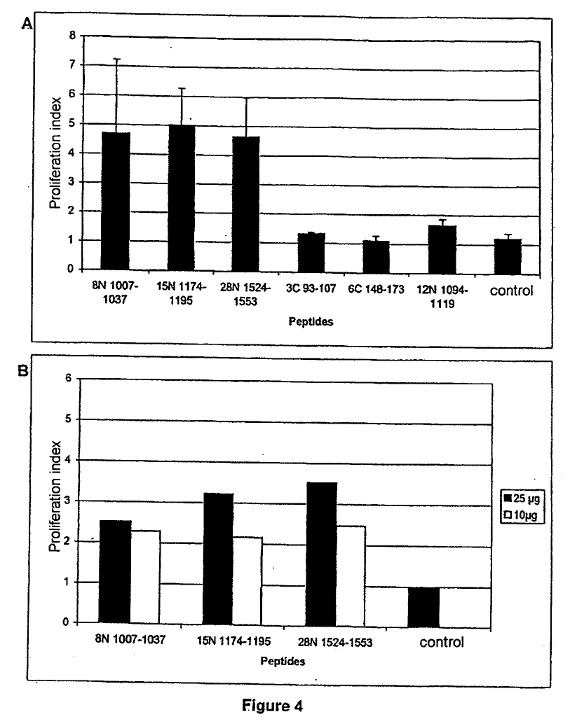

FIG. 4 illustrates the in vivo immunogenicity of 3 peptides of the NS3 protein exhibiting high affinity for HLA-DR1 (8N 1007-1037, 15N 1174-1195 (15N) and 28N 1524-1553), measured by means of a proliferation assay using splenocytes from mice transgenic for the human HLA-DR1 molecules, immunized beforehand with each of the peptides (FIG. 4A) or with a mixture of these peptides (FIG. 4B). The peptides 3C 93-107, 6C 148-173 and 12N 1094-1119 which have low affinity for HLA-DR1 are used as a control. The values correspond to the splenocyte proliferation index;

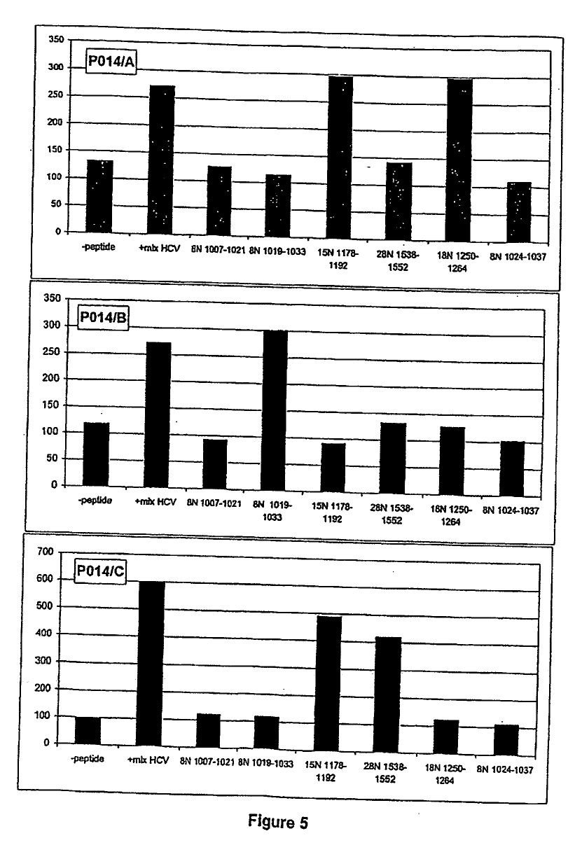

FIG. 5 illustrates the in vitro immunogenicity of 6 peptides of the NS3 protein exhibiting high affinity for at least 4 different HLA II molecules that are predominant in the Caucasian population (8N 1007-1021, 8N 1019-1033, 15N 1178-1193, 28N 1538-1552, 18N 1250-1264 and 8N 1024-1037), measured by means of an ELISPOT assay, using 3 CD4+ T lymphocyte lines derived from a seronegative individual (P014/A, P014/B and P014/Q); the T lymphocytes were stimulated beforehand in vitro with dendritic cells from this same individual, loaded with the mixture of peptides, and then the number of T lymphocytes secreting IFN-γ was measured in the presence of non-loaded dendritic cells derived from the same individual and in the presence of these peptides alone or as a mixture (mix HCV) or else in the absence of peptides (-peptide). The values indicated correspond to the number of T lymphocytes secreting IFN-γ.

It should be clearly understood, however, that these examples are given only by way of illustration of the subject of the invention, of which they in no way constitute a limitation.

### EXAMPLE 1

### Determination of the Conditions for the Peptide/HLA II Molecule Binding Assays

1) Peptides

All the peptides are synthesized according to the Fmoc strategy in parallel synthesis on solid phase, purified by HPLC and controlled by mass spectrometry (ES-MS).

A) First Series of Peptides

The HLA II molecule-binding activity of the peptides of the C and NS3 proteins of HCV was tested using 25 large fragments (between 15 and 34 amino acids), chosen according to two criteria:

presence of several aromatic or hydrophobic residues which are the main anchoring residues for HLA-DR and HLA-DP molecules, and

good probability of being able to be synthesized.

The sequences of the selected peptides, derived from genotype 1a, are given in table III below.

TABLE III
Sequences of the first series of peptides

| | | | |
|---|---|---|---|
| Name | Sequence | Size | Number |

| | | | |
|---|---|---|---|
| IC 19-47 | PQDVKFPGGGQIVGGVYLLPRRGPRLGVR | 29 | SEQ ID NO: 1 |
| 2C 31-57 | VGGVYLLPRRGPRLGVRATRKTSERSQ | 27 | SEQ ID NO: 2 |
| 3C 93-107 | WAGWLLSPRGSRPSW | 15 | SEQ ID NO: 3 |
| 4C 104-133 | RPSWGPTDPRRRNLGKVIDTLTCGFADL | 30 | SEQ ID NO: 4 |
| 5C 127-149 | TAGFADLMGYIPLVGAPLGGAAR | 23 | SEQ ID NO: 5 |
| 6C 148-473 | ARALAHGVRVLEDGVNYATGNLPGAS | 26 | SEQ ID NO: 6 |
| 8N 1007-1037 | GREILLGPADGMVSKGWRLLAPITAYAQQTR | 31 | SEQ ID NO: 7 |
| 9N 1036-1055 | TRGLLGCIITSLTGRDKNQV | 20 | SEQ ID NO: 8 |
| 10N 1052-1072 | KNQVEGEVQIVSTAAQTFLAT | 21 | SEQ ID NO: 9 |
| 11N 1076-1093 | GVAWTVYHGAGTRTIASP | 18 | SEQ ID NO: 10 |
| 12N 1094-1119 | KGPVIQMYTNVDQDLVGWPAPQGSRS | 26 | SEQ ID NO: 11 |
| 13N 1127-1153 | SSDLYLVTRHADVIPVRRRGDSRGSLL | 27 | SEQ ID NO: 12 |
| 14N 1149-1172 | RGSLLSPRPISYLKGSSGGPLLAP | 24 | SEQ ID NO: 13 |
| 15N 1174-1195 | GHAVGIFRAAVCTRGVAKAVDP | 22 | SEQ ID NO: 14 |
| 16N 1190-1212 | AKAVDFIPVENLETTMRSPVFTD | 23 | SEQ ID NO: 15 |
| 17N 1206-1239 | RSPVFTDNSSPPVVPQSFQVAHLHAPTGSGKSTK | 34 | SEQ ID NO: 16 |
| 18N 1246-1275 | AQGYKVLVLNPSVAATLGFGAYMSKAHGID | 30 | SEQ ID NO: 17 |
| 19N 1275-1304 | DPNIRTGVRTTTTGSPITYSTYGKFLADGG | 30 | SEQ ID NO: 18 |
| 22N 1362-1387 | IEEVALSTTGEIPFYGKAIPLEVIKG | 26 | SEQ ID NO: 19 |
| 23N 1377-1403 | GKAIPLEVIKGGRHLIFCHSKKKADEL | 27 | SEQ ID NO: 20 |
| 24N 1404-1432 | AAKLVALGINAVAYYRGLDVSVIPTSGDV | 29 | SEQ ID NO: 21 |
| 26N 1456-1481 | TAVTQTVDFSLDPTFTIETITLPQDA | 26 | SEQ ID NO: 22 |
| 27N 1495-1513 | KPGIYRFVAPGERPSGMFD | 19 | SEQ ID NO: 23 |
| 28N 1524-1553 | GAAWYELTPAETTVRLRAYMNTPGLPVAQD | 30 | SEQ ID NO: 24 |
| 29N 1552-1583 | QDHLEFWEGVFTGLTHIDAHFLSQTKQSGENL | 32 | SEQ ID NO: 25 |

*the alanine residues corresponding to the substitution of a cysteine residue of the sequence of the HCV polyprotein are indicated in bold.

B) Second Series of Peptides

All the peptides of the first series that bind to at least 6 HLA II molecules and some peptides that bind to at least 5 HLA II molecules were cut up into peptides of 15 amino acids in order to identify more precisely the zones of interaction (peptides 8N, 9N, 10N, 15N, 16N, 18N, 19N, 23N, 27N, 28N, 29N, 1C, 2C and 5C). Only peptide 26 was not studied, because of the difficulty in synthesizing it. Peptide 6C, which overlaps substantially with peptide 5C and which, in combination with the latter, binds to 6 HLA II molecules, was also studied.

The sequence of all these peptides is given in table IV and FIG. 1.

TABLE IV
Sequences of the second series of peptides

| | | |
|---|---|---|
| Name | Sequence | Number |

| | | |
|---|---|---|
| 8N 1007-1037 | GREILLGPADGMVSKGWRLLAPITAYAQQTR | SEQ ID NO: 7 |
| 8N 1007-1021 | GREILLGPADGMVSK | SEQ ID NO: 26 |
| 8N 1011-1025 | LLGPADGMVSKGWRL | SEQ ID NO: 27 |
| 8N 1015-1029 | ADGMVSKGWRLLAPI | SEQ ID NO: 28 |
| 8N 1019-1033 | VSKGWRLLAPITAYA | SEQ ID NO: 29 |
| 8N 1020-1034 | SKGWRLLAPITAYAQ | SEQ ID NO: 30 |
| 8N 1024-1037 | RLLAPITAYAQQTR | SEQ ID NO: 31 |
| 15N 1174-1195 | GHAVGIFRAAVCTRGVAKAVDF | SEQ ID NO: 14 |
| 15N 1174-1188 | GHAVGIFRAAVCTRG | SEQ ID NO: 32 |
| 15N 1178-1192 | GIFRAAVCTRGVAKA | SEQ ID NO: 33 |
| 15N 1181-1195 | RAAVCTRGVAKAVDF | SEQ ID NO: 34 |
| 28N 1524-1553 | GAAWYELTPAETTVRLRAYMNTPGLPVAQD | SEQ ID NO: 24 |
| 28N 1524-1538 | GAAWYELTPAETTVR | SEQ ID NO: 35 |
| 28N 1528-1542 | YELTPAETTVRLRAY | SEQ ID NO: 36 |
| 28N 1529-1543 | ELTPAETTVRLRAYM | SEQ ID NO: 37 |
| 28N 1534-1548 | ETTVRLRAYMNTPGL | SEQ ID NO: 38 |
| 28N 1538-1552 | RLRAYMNTPGLPVAQ | SEQ ID NO: 39 |
| 28N 1540-1553 | RAYMNTPGLPVAQD | SEQ ID NO: 40 |
| 18N 1246-1275 | AQGYKVLVLNPSVAATLGFGAYMSKAHGID | SEQ ID NO: 17 |
| 18N 1246-1260 | AQGYKVLVLNPSVAA | SEQ ID NO: 41 |
| 18N 1250-1264 | KVLVLNPSVAATLGF | SEQ ID NO: 42 |
| 18N 1255-1269 | NPSVAATLGFGAYMS | SEQ ID NO: 43 |
| 18N 1261-1275 | TLGFGAYMSKAHGID | SEQ ID NO: 44 |
| 1C 19-47 | PQDVKFPGGGQIVGGVYLLPRRGPRLGVR | SEQ ID NO: 1 |
| 2C 31-57 | VGGVYLLPRRGPRLGVRATRKTSERSQ | SEQ ID NO: 2 |
| IC 19-33 | PQDVKFPGGGQIVGG | SEQ ID NO: 45 |
| IC 27-41 | GGGQIVGGVYLLPRRG | SEQ ID NO: 46 |
| 2C 31-45 | GGVYLLPRRGPRLGV | SEQ ID NO: 47 |
| 2C 43-57 | RLGVRATRKTSERSQ | SEQ ID NO: 48 |
| 5C 127-149 | TAGFADLMGYIPLVGAPLGGAAR | SEQ ID NO: 5 |
| 6C 148-173 | ARALAHGVRVLEDGVNYATGNLPGAS | SEQ ID NO: 6 |
| 5C 127-141 | TAGFADLMGYIPLVG | SEQ ID NO: 49 |
| 5C 131-145 | ADLMGYIPLVGAPLG | SEQ ID NO: 50 |
| 5C 134-148 | MGYIPLVGAPLGGA | SEQ ID NO: 51 |
| C 141-155 | GAPLGGAARALAHG | SEQ ID NO: 52 |
| 6C 148-163 | ARALAHGVRVLEDGV | SEQ ID NO: 53 |
| 6C 152-166 | AHGVRVLEDGVNYAT | SEQ ID NO: 54 |
| 6C 159-173 | EDGVNYATGNLPGAS | SEQ ID NO: 55 |
| 16N 1190-1212 | AKAVDFIPVENLETTMRSPVFTD | SEQ ID NO: 15 |
| 16N 1190-1204 | AKAVDFIPVENLETT | SEQ ID NO: 56 |
| 16N 1192-1206 | AVDFIPVENLETTMR | SEQ ID NO: 57 |
| 16N 1196-1210 | IPVENLETTMRSPVF | SEQ ID NO: 58 |
| 16N 1199-1212 | ENLETTMRSPVFTD | SEQ ID NO: 59 |
| 10N 1052-1072 | KNQVEGEVQIVSTAAQTFLAT | SEQ ID NO: 9 |
| 10N 1052-1066 | KNQVEGEVQIVSTAA | SEQ ID NO: 60 |
| 10N 1056-1070 | EGEVQIVSTAAQTFL | SEQ ID NO: 61 |
| 23N 1377-1403 | GKAIPLEVIKGGRHLIFCHSKKKADEL | SEQ ID NO: 20 |
| 23N 1377-1391 | GKAIPLEVIKGGRHL | SEQ ID NO: 62 |
| 23N 1381-1395 | PLEVIKGGRHLIFCH | SEQ ID NO: 63 |
| 23N 1383-1397 | EVIKGGRHLIFCHSK | SEQ ID NO: 64 |
| 23N 1389-1403 | RHLIFCHSKKKADEL | SEQ ID NO: 65 |
| 27N 1495-1513 | KPGIYRFVAPGERPSGMFD | SEQ ID NO: 23 |
| 27N 1495-1509 | KPGIYRFVAPGERPS | SEQ ID NO: 66 |
| 27N 1500-1513 | RFVAPOERPSGMFD | SEQ ID NO: 67 |
| 29N 1552-1583 | QDHLEFWEGVFTGLTHIDAHFLSQTKQSGENL | SEQ ID NO: 25 |
| 29N 1554-1568 | HLEFWEGVFTGLTHI | SEQ ID NO: 68 |
| 29N 1565-1579 | LTHIDAHFLSQTKQS | SEQ ID NO: 69 |
| 29N 1559-1573 | EGVFTGLTHIDAHFL | SEQ ID NO: 70 |
| 29N 1563-1577 | TGLTHIDAHFLSQTK | SEQ ID NO: 71 |
| 29N 1569-1583 | DAHFLSQTKQSGENL | SEQ ID NO: 72 |
| 9N 1036-1055 | TRGLLGCITTSLTGRDKNQV | SEQ ID NO: 8 |
| 9N 1036-1050 | RGLLGCITTSLTGRD | SEQ ID NO: 73 |
| 9N 1041-1055 | GCITTSLTGRDKNQV | SEQ ID NO: 74 |
| 19N 1275-1304 | DPNIRTGVRTITTGSPITYSTYGKFLADGG | SEQ ID NO: 18 |
| 19N 1275-1289 | DPNIRTGVRTITTGS | SEQ ID NO: 83 |
| 19N 1279-1293 | RTGVRTITTGSPITY | SEQ ID NO: 84 |
| 19N 1283-1297 | RTITTGSPITYSTYG | SEQ ID NO: 85 |
| 19N 1290-1304 | PITYSTYGKFLADGG | SEQ ID NO: 86 |

2) HLA II Molecules
a) Choice of Alleles

12 HLA II molecules (10 HLA-DR molecules and 2 HLA-DP molecules) that are the most abundant in the French population, and for which the allelic frequencies are characteristic of the Caucasian population, were selected (table VIII):

HLA-DR Molecules in which the β-Chain is Encoded by the DR1 Gene

These are the HLA-DR1, -DR3, -DR4, -DR7, -DR11, -DR13 and -DR15 molecules in which the β-chain is encoded by the alleles of the DRB1 locus whose frequency exceeds 5% in the French population: DRB1*0101, DRB1*0301, DRB1*0401, DRB1*0701, DRB1*1101, DRB1*1301 and DRB1*1501, which represent, by themselves, 64% of the population. These same alleles are the HLA-DR alleles that are the most abundant in the other Caucasian populations. Their frequency ranges between 53% (in Spain) and 82% (in Denmark). For the United States and Canada, they represent, respectively, 58 and 55% of the DR alleles of the population.

HLA-DR Molecules in which the β-Chain is not Encoded by the DR1 Gene

These are the HLA-DRB3, -DRB4 and -DRB5 molecules in which the β-chain is encoded by the alleles that are the most common in the French population: HLA-DRB3*0101 (9.2%), HLA-DRB4*0101 (28.4%) and HLA-DRB5*0101 (7.9%). These molecules cover, by themselves, 45% of the allelic frequency.

HLA-DP Molecules

These are the HLA-DP4 molecules which group together the molecules encoded by the DPB1*0401 and DPB1*0402 alleles. These DP4 molecules are the HLA II molecules that are the most abundant in Europe and in the United States. Their allelic frequency is in fact 40 and 11%, respectively, which means that either one of them is found in approximately 76% of individuals. The peptides present in a protein sequence, and which bind all these molecules, therefore include the CD4⁺ T epitopes of the majority of the population.

b) Purification of the HLA II Molecules

The HLA II molecules are purified by immunoaffinity from various homozygous lines of human B lymphocytes transformed with the Epstein Barr virus (EBV).

The origin of the EBV lines and the various alleles which characterize them are described in tables V and VI below.

TABLE V
DR specificity of the EBV lines

| | | | Other |
|---|---|---|---|
| | | DRB1 | DRB |
| Lines | Specificity | allele | alleles |
| LG2* | DR1 | DRB1*0101 | |
| HOM2 | DR1 | DRB1*0101 | |
| SCHU | DR15 | DRB1*1501 | DRB5*0101 |
| MAT* | DR3 | DRB1*0301 | DRB3*0101 |
| STEILIN | DR3 | DRB1*0301 | DRB3*0101 |
| BOLETH° | DR4 | DRB1*0401 | DRB4*0101 |
| PREISS° | DR4 | DRB1*0401 | DRB4*0101 |
| PITOUT° | DR7 | DRB1*0701 | DRB4*0101 |
| SWEIG° | DR11 | DRB1*1101 | DRB3*0202 |
| HHKB°° | DR13 | DRB1*1301 | DRB3*0101 |

°Strang et al., J. Gen. Virol., 1990, 71, 423,
°°Tsukui et al., Cancer Res., 1996, 56, 3967

TABLE VI
EBV lines expressing DP4

| | DP | DPA1 | DPB1 | |
|---|---|---|---|---|
| Lines | specificity | allele | allele | Reference |
| HOM2 | DP4 | DPA1*0103 | DPB1*0401 | ° |
| BOLETH | DP4 | DPA1*0103 | DPB1*0401 | ° |
| PITOUT | DP4 | DPA1*0103 | DPB1*0401 | SOUTHWOOD et |
| | | | | al., J. Immunol., |
| | | | | 1998, 160, 3363- |
| | | | | 3373 |
| HHKB | DP4 | DPA1*0103 | DPB1*0401 | DAVENPORTH et |
| | | | | al, P.N.A.S., 1995, |
| | | | | 92, 6567. |
| SHU | DP4 | DPA1*0103 | DPB1*0402 | ° |
| MLF | DP4 | DPA1*0103 | DPB1*0402 | ° |
| BM92 | DP4 | DPA1*0103 | DPB1*0402 | ° |

°The origin of the lines is described on the European Cell Culture Collection internet site (http://fuseiv.co.uk/camr/.)

The HLA-DR and HLA-DP molecules are immunopurified by means of the monoclonal antibodies L243 (Smith et al., P.N.A.S., 1982, 79, 608-612) and B7/21 (Watson et al., Nature, 1983, 304, 358-361) respectively, according to the protocols described in Texier et al. (J. Immunol., 2000, 164, 3177; Eur. J. Immunol., 2001, 31, 1837).

3) HLA II/Peptide Binding Assays

a) Principle of the Assays

The assays for binding of the peptides to the HLA-DP and HLA-DR molecules are competition assays with immunoenzymatic visualization, that are derived from those developed for HLA-DR molecules (HLA-DR1: Marshall et al., J. Immunol., 1994, 152, 4946-; HLA-DR1, -DR2, -DR3, -DR4, -DR7, -DR11 and -DR13: patent application FR 99 0879 and Texier et al., mentioned above).

The binding assays are carried out in the following way: the HLA-DR or HLA-DP molecules are diluted in polypropylene 96-well plates, in a 10 mM phosphate buffer containing 150 mM NaCl, 1 mM dodecyl maltoside (DM), 10 mM citrate and 0.003% thimerosal, at a pH and a dilution that are suitable for each molecule. A biotinylated tracer peptide (table VIII) is added at a given concentration, as are several concentrations of test peptides (competitor peptide). At the end of the incubation at 37° C. (between 24 and 72 hours according to the molecules), the samples are neutralized with 50 μl of 450 mM Tris HCl buffer, pH 7.5, containing 0.003% thimerosal, 0.3% BSA and 1 mM DM. They are then transferred onto maxisorp (96-well) ELISA plates onto which the anti-DP or anti-DR antibodies have been preadsorbed. The incubation of the samples on these plates is carried out for two hours at ambient temperature. Washes are carried out between each step, in 0.1M Tris HCl buffer, pH 7.5, containing 0.05% Tween-20. The biotinylated peptide bound to the HLA II molecules is detected by adding 100 μl/well of the streptavidin-alkaline phosphatase conjugate (45 minutes) diluted to 1/2000 in the 10 mM Tris buffer, pH 7, containing 0.15M NaCl, 0.05% Tween 20, 0.2% BSA and 0.003% thimerosal, and then 200 μl/well of the 4-methylumbelliferyl phosphate (MUP) substrate at a concentration of 100 μM, in 0.05M NaHCO₃ buffer, pH 9.8, containing 1 mM MgCl₂. The fluorescence emission by the product of the enzyme reaction is measured at 450 nm after excitation at 365 nm. The maximum binding is determined by incubating the biotinylated tracer peptide with the HLA II molecule in the absence of competitor peptide. The binding specificity is controlled by adding an excess of nonbiotinylated peptide. The background noise obtained does not significantly differ from that obtained by incubating the biotinylated peptide without the HLA II molecules. The results are expressed in the form of the concentration of competitor peptide which inhibts 50% of the maximum binding of the biotinylated tracer peptide (IC₅₀).

b) Assay Conditions and Sensitivity

For each binding assay, the concentration of HLA II molecules, the concentration of the tracer peptide, the incubation pH and the incubation time were optimized as specified in table VII below.

TABLE VII
Conditions for the HLA II molecule-binding assays

| | | | Tracer | | |
|---|---|---|---|---|---|
| | | | concen- | Opti- | Incubation |
| | | | tration | mum | time |
| Alleles | Dilution | Tracers | (nM) | pH | (h) |
| DRB1*0101 | 1/40 to 1/400 | HA 306-318 | 1 | 6 | 24 |
| DRB1*0301 | 1/10 to 1/40 | MT 2-16 | 200 | 4.5 | 72 |
| DRB1*0401 | 1/20 to 1/100 | HA 306-318 | 30 | 6 | 24 |
| DRB1*0701 | 1/20 to 1/100 | YKL | 10 | 5 | 24 |
| DRB1*1101 | 1/20 to 1/100 | HA 306-318 | 20 | 5 | 24 |
| DRB1*1301 | 1/10 to 1/40 | B1 21-36 | 200 | 4.5 | 72 |
| DRB1*1501 | 1/10 to 1/100 | A3 152-166 | 10 | 4.5 | 72 |
| DRB5*0101 | 1/10 to 1/100 | HA 306-318 | 10 | 5.5 | 24 |
| DRB3*0101 | 1/10 to 1/100 | Lol 191-120 | 10 | 5.5 | 24 |
| DRB4*0101 | 1/10 to 1/100 | E2/E168 | 10 | 5 | 72 |
| DBP1*401 | 1/20 to 1/400 | Oxy 271-287 | 10 | | 24 |
| DPB1*402 | 1/20 to 1/100 | Oxy 271-287 | 10 | | 24 |

The sensitivity of each assay is reflected by the IC₅₀ values observed with the nonbiotinylated peptides which correspond to the tracers, and the results obtained are given in table VIII below.

TABLE VIII

Sensitivity of the assays for binding to
the HLA II molecules predominant in the Caucasian
population

| | | | | |
|---|---|---|---|---|
| | | | Sequences | IC₅₀ |
| Alleles | Frequency | Peptides | (SEQ ID NO: 75 to 82) | (nM) |

| | | | | |
|---|---|---|---|---|
| DRB1*0101 | 9.3 | HA 306-318 | PKYVKQNTLKLAT | 31 |
| DRB1*0401 | 5.6 | HA 306-318 | PKYVKQNTLKLAT | 44 |
| DRB1*1101 | 9.2 | HA 306-318 | PKYVKQNTLKLAT | 38 |
| DRB1*0701 | 14.0 | YKL | AAYAAAKAAALAA | 34 |
| DRB1*0301 | 10.9 | MT2-16 | AKTIAYDEEARRGLE | 100 |
| DRB1*1301 | 6.0 | B1 21-36 | TERVRLVTRHIYNREE | 330 |
| DRB1*1501 | 8.0 | A3 152-166 | EAEQLRRAYLDGTGVE | 14 |
| DRB5*0101 | 7.9 | HA 306-318 | PKYVKQNWKLAT | 6.5 |
| DRB3*0101 | 9.2 | Lo1 191-120 | ESWGAVWRIDTPDKLTGPFT | 5 |
| DRB4*0101 | 28.4 | E2/E168 | AGDLLAIETDKATI | 2 |
| DPB1*401 | 40 | Oxy 271-287 | EKKYFAATQFEPLAARL | 10 |
| DPB1*402 | 11 | Oxy 271-287 | EKBVYFAATQPEPLAARL | 10 |

The frequencies indicated, derived from Colombani (HLA: immune functions and medical applications. 1993. Publishers John Libbey Eurotext), which are the allelic frequencies in France, are representative of those for the Caucasian population.

### EXAMPLE 2

### Binding Activities of the C and NS3 Peptides With Respect to the HLA II Molecules that are Predominant in the Caucasian Population

1) First Series of Peptides (FIG. 2)

The binding activity of the long peptides as defined in example 1, with respect to the 12 HLA II molecules that are predominant in the Caucasian population (HLA-DR1, -DR3, -DR4, -DR7, -DR11, -DR13, -DR15, -DRB3, -DRB4, -DRB5, -DP401 and -DP402; table VIII), was measured under the conditions specified in example 1.

The results given in FIG. 2 show that each peptide binds with good affinity (IC₅₀<1000 nM) to at least one HLA II molecule and that several peptides bind with good affinity to several HLA II molecules. Among the latter peptides, the peptides 26N, 8N, 28N, 1C, 10N, 15N, 19N, 29N, 2C, 16N, 18N, 23N, 27N, 5C, 9N, 11N, 13N, 14N, 17N, 24N, 4C and 22N bind with good affinity to at least 4 different HLA II molecules.

2) Second Series of Peptides (FIG. 3 and Table IX)

The results given in FIG. 3 and table IX make it possible to specify the zones of interaction of the peptides with the various HLA II molecules.

TABLE IX
Binding activities of peptides 9N and 29N with respect to the HLA
II molecules that are predominant in the Caucasian population

| | DR1 | DR3 | DR4 | DR7 | DR11 | DR13 | DR15 | DRB3 | DRB4 | DRB5 |
|---|---|---|---|---|---|---|---|---|---|---|
| 9N 1036-1055 | 51 | >100000 | 950 | 2600 | 700 | >100000 | 5500 | | 1000 | 53 |
| 9N 1036-1050 | 16 | >100000 | 350 | 2500 | 1100 | >100000 | 1550 | | 300 | 2000 |
| 9N 10411055 | 190 | 62500 | 2900 | 25000 | 2600 | >100000 | 525 | | 65000 | 11 |
| 29N 1552-1583 | 170 | 6750 | 4000 | 750 | 90 | 27333 | 60 | | 25000 | 333 |
| 29N 1559-1573 | 29 | >100000 | 3000 | 18 | 7 | >100000 | 305 | | >100000 | 32 |
| 29N 1563-1577 | 8 | 3900 | 2100 | 16 | 817 | 3250 | 13 | | 40000 | 12 |
| 29N 1569-1583 | 950 | >100000 | 10500 | 3000 | 22 | >100000 | 2800 | | >100000 | 21000 |

### EXAMPLE 3

### Immunogenicity OF NS3 Peptides In Vivo in Mice that are Transgenic for an HLA II Molecule

The ability of the peptides having good affinity for the HLA II molecules, as defined above, to induce an immune response was evaluated by means of an in vitro proliferation assay in mice transgenic for HLA-DR1 molecules (Wilkinson et al., Infect. Immunity, 1999, 67, 1501-; Rosloniec et al., J. Exp. Med., 1997, 185, 1113-), pre-immunized with these peptides.

More precisely, the protocol used is as follows: 25 μg of each of the peptides 8N 1007-1037, 15N 1174-1195, 28N 1524-1553, 3C 93-107, 6C 148-173 and 12N 1094-1119, separately, or else 10 μg and 25 μg of a mixture of the same 8N, 15N and 28N peptides, were emulsified in montanide and injected subcutaneously into a group of 5 mice. 15 days later, a second injection was given under the same conditions. Ten days after the second injection, the animals were sacrificed and their spleen was removed. The splenocytes were cultured in HL-1 medium (Biowhittaker) supplemented with 1% of mouse serum, in the presence of 5 μg/ml of each of the peptides used for the injection, or in the absence of peptide. After 4 days of culture, 1 μCi of tritiated thymidine (Amersham, Life Technologies) was added for 16 h. The tritiated thymidine incorporation was then measured using a beta-counter (Microbeta 1450, Perkin Elmer). The results are expressed as a proliferation index (number of CPM in the presence of peptide/number of CPM in the absence of peptide).

The results show that the peptides 8N 1007-1037, 15N 1174-1195 and 28N 1524-1553 which have a strong capacity for binding to the HLA-DR1 molecule (IC₅₀ of, respectively, 2, 3 and 2 nM) induce a strong immune response in these mice, whereas the peptides 3C93-107, 6C148-173 and 12N1094-1119 which have a weak capacity for binding to this molecule (IC₅₀ of, respectively, 6333, 10 000 and >10 000 nM) do not induce any effect (FIG. 4A). These results also show that the injection of a mixture of peptides has no harmful effect on the immune response and makes it possible to induce a strong response against each of the peptides of the mixture (FIG. 4B).

These results confirm that the peptides selected by means of the HLA II molecule-binding assay are capable of inducing a strong CD4+ response in the immunized individuals. They therefore indicate that the selected peptides can be used in immunogenic compositions for immunization against hepatitis C.

### EXAMPLE 4

### Immunogenicity of NS3 Peptides In Vitro

The ability of the peptides having good affinity for the HLA II molecules to induce stimulation of specific T lymphocytes in vitro was evaluated using blood samples from individuals seronegative for HCV. This involves evaluating the ability to recruit CD4+ precursor lymphocytes although they are, in a naïve individual, at very low frequency, i.e. to carry out an immunization in vitro by means of these peptides.

a) Materials and Methods

Peripheral blood mononuclear cells (PBMCs) were separated on a Ficoll gradient. The PBMCs were then cultured in AIM V medium (Life Technologies) and incubated in flasks, in an incubator at 37° C. in the presence of 5% of CO₂. After overnight incubation, the non-adherent cells were recovered and loaded onto an LS+column (Miltenyi), and the CD4+ T lymphocytes thus purified were then frozen. The adherent cells were incubated for 5 days in AIM V medium containing 1000 U/ml of GM-CSF and 1000 U/ml of IL-4, and the cells that had differentiated into dendritic cells were then cultured for 2 days in the presence of 1 μg/ml of LPS, 1000 U/ml of IL-4 and 1000 U/ml of GM-CSF, so as to induce their maturation.

The mature dendritic cells (100 000 cells/well) were then incubated with a mixture of peptides (10 μg of each peptide), for 5 hours at 37° C. The mature dendritic cells were subsequently washed and then incubated, in the presence of the CD4+ T lymphocytes (100 000 cells/well) thawed beforehand, in medium containing 1000 U/ml of IL-6 and 10 ng/ml of IL-12. After 7 days, the culture was restimulated a first time by means of mature dendritic cells that had been thawed and loaded with the mixture of peptides beforehand, in medium containing IL-2 (10 U/ml) and IL-7 (5 ng/ml). 7 days later, the culture was restimulated, in a similar manner, in a medium containing no IL-2. After two further stimulations, under the above conditions, the cells were assayed by ELISPOT, in the following way:

Anti-IFNγ antibodies (1-D1K, Mabtech) diluted to 1 μg/ml in PBS buffer were adsorbed onto nitrocellulose plates (Millipore) for 1 hour at 37° C. The plates were then washed with PBS and then saturated with RPMI medium containing 10% FCS (100 μl/well), for 2 h at 37° C. The prethawed mature dendritic cells (10 000 cells/well) and 10 000 lymphocytes to be tested were then added to the plates and incubated for 24 h at 37° C., in the presence or absence of a single peptide or of a mixture of peptides. After washing with PBS buffer and then with PBS/0.05% Tween, 100 μl of biotin-conjugated anti-IFNγ secondary antibody (7-B6-1-biotin, Mabtech), diluted to 1 μg/ml in PBS containing 0.05% Tween 20 and 1% BSA, were added to each well. After incubation for one hour, the plates were washed again, and incubated with 100 μl/well of Extravidin-AKP (No. 2636, SIGMA), diluted to 1/5000 in PBS containing 0.05% Tween 20 and 1% BSA. The immunoenzymatic reaction was then visualized by means of the kit No. 170-6432 (Biorad).

b) Results

The PBMCs of a seronegative patient (P014) were assayed according to the protocol as defined above, with the mixture of the following peptides: 8N 1007-1021, 8N 1019-1033, 15N 1178-1192, 28N 1538-1552, 18N 1250-1264 and 8N 1024-1037.

After 4 restimulations, an ELISPOT was carried out on various wells. Three specific lines could be demonstrated (FIG. 5). These three lines respond in the presence of the peptide mixture (mix) but not in the absence of the mixture. Each line responds to at least one of the peptides of the mixture. The P014/A line responds to the 15N 172-186 peptiode and to the 18N 244-258 peptide (FIG. 5A). The P014/B line responds to the 8N 13-27 peptide (FIG. 5B) and the P014/C line responds to the 15N 172-186 and 28N 532-546 peptides (FIG. 5C).

### EXAMPLE 5

### Detection of HCV-Specific T Lymphocytes Using the C and NS3 Peptides

The ability of the peptides having high affinity for the HLA class II molecules, as defined above, to detect T lymphocytes specific for the hepatitis C virus (HCV) was tested in vitro by assaying the cytokines IFN-γ, IL-2, IL-4 and IL-10 produced by the T lymphocytes derived from PBMCs from patients chronically infected with HCV.

More precisely, the peripheral blood mononuclear cells (PBMCs) of patients chronically infected with HCV were separated on a Ficoll gradient, using a heparinized blood sample. The isolated PBMCs were then cultured at 37° C. in 96-well plates containing complete (10% human serum, 100 IU/ml penicillin and 10 μg/ml streptomycin) RPMI 1640 medium (Gibco), alone or in the presence:

of a mixture of C peptides 19-47 and 31-57, of a mixture of C peptides 127-149 and 148-173 or of NS3 peptides 1007-1037, 1036-1055, 1074-1195, 1190-1212, 1377-13403, 1524-1553, 1552-1583, each at a concentration of 10 μg/ml,

of a mixture of all these peptides, each at a concentration of 110 μ/ml, 55 μg/ml or 11 μg/ml, or

of phytohemagglutinin (PHA) or of tetanus toxin (TT), each at a concentration of 2.5 μg/ml.

The culture supernatants were harvested at 24 h or 48 h and stored at −80° C. until analysis.

The cytokines possibly present in the supernatants were assayed in the following way:

96-well plates (Maxisorp, NUNC) were coated with 100 μl of anti-IFNγ, IL-2, IL-4 and IL-10 antibodies (Becton Dickinson, 2 μg/ml) in 0.1 M carbonate buffer, pH 8.6, and incubated for 2 h at 37° C. After three successive washes with PBS buffer containing 0.05% of Tween 20, the plates were saturated with 200 μl of PBS containing 3% of bovine serum albumin (BSA, SIGMA), for 2 h at ambient temperature. 50 μl of culture supernatant (in duplicate), and also a standard range of recombinant human cytokines (rH-IFN-γ, rH-IL-2, rH-IL-4 and rH-II-10; Becton Dickinson) were then added to each well and the plates were incubated for 12 h at +4° C. After three successive washes with PBS buffer containing 0.05% of Tween 20, 100 μl of anti-IFN-γ, IL-2, IL-4 and IL-10 secondary antibodies (Becton Dickinson, 1 μg/ml) in PBS buffer containing 1% of BSA were added to the wells and the wells were then incubated for 1 h at ambient temperature. After four successive washes with PBS buffer containing 0.05% of Tween 20, 100 μl of streptavidin peroxidase (SIGMA) diluted to 1/10 000 in PBS buffer containing 0.1% BSA were added to the wells, and the plates were then incubated for 30 minutes at ambient temperature. After four successive washes with PBS buffer containing 0.05% of Tween 20, 100 μl of ortho-phenylenediamine (OPD, SIGMA, 400 μg/ml) in 50 mM citrate buffer were added to the wells and the plates were incubated at ambient temperature, and then the reaction was stopped by adding 50 μl of 2N HCl. The optical density was then measured at 540 nm and the quantitative analysis of the cytokines was carried out using the Deltasoft program (DS3-1.518F/1994 E. Berchtold & Biometallics Inc.). The results, expressed in pg/ml, are given in table X.

TABLE X
Assay of cytokines produced by the T lymphocytes of chronic infected
patients, after stimulation with the C and NS3 peptides, alone or
as a mixture

| | | | | | | | | | | | | | | |
|---|---|---|---|---|---|---|---|---|---|---|---|---|---|---|
| | | | | | | 1C + | 5C + | | | | | | | |
| — | PHA | TT | M110 | M55 | M11 | 2C | 6C | 8N | 9N | 15N | 16N | 23N | 28N | 29N |

patient 815

| | | | | | | | | | | | | | | | |
|---|---|---|---|---|---|---|---|---|---|---|---|---|---|---|---|
| IL2 | — | 1002 | 610 | — | — | — | — | — | — | — | — | 72 | 58 | | |
| IFNγ | — | 1979 | — | — | — | — | — | — | — | — | — | — | — | — | — |
| IL4 | — | 5638 | — | — | — | — | — | — | — | — | — | — | — | — | — |
| IL10 | — | 497 | — | 79 | 27 | — | 11* | | — | 156 | — | 123 | 63* — | — | — |
| patient 829 | | | | | | | | | | | | | | | |
| IL2 | — | NT | — 6 | 3 | 3 | 3 | — | — | — | — | — | — | — | — | — |
| IFNγ | — | NT | 441 | — | — | — | — | — | — | — | — | — | — | — | — |
| IL4 | — | NT | — | — | — | — | — | — | — | — | — | — | — | — | — |
| IL10 | — | NT | — | — | — | — | — | — | — | — | — | — | — | 67 | — |
| patient 342 | | | | | | | | | | | | | | | |
| IL2 | — | 125 | 22 | 20 | — | — | — | — | — | — | — | — | — | — | — |
| IFNγ | — | — | — | — | — | — | — | — | — | — | — | — | — | — | — |
| IL4 | — | — | — | — | — | — | — | — | — | — | — | — | — | — | — |
| IL10 | NT | | NT | NT | NT | NT | NT | NT | NT | NT | NT | NT | NT | NT | NT |
| patient 862 | | | | | | | | | | | | | | | |
| IL2 | — | 60 | — | — 4 | 8* | — 6 | — | — | — | 29.1 | — | — | — | — | — |
| IFNγ | — | 9902 | — | — | — | — | — | — | — | — | — | — | — | — | — |
| IL4 | — | 9 | — | — | — | — | — | — | — | — | — | — | — | — | 57 |
| IL10 | NT | NT | NT | NT | NT | NT | NT | NT | NT | NT | NT | NT | NT | NT | NT |
| patient 093 | | | | | | | | | | | | | | | |
| IL2 | — | 1402* | 171 | 7 | 8 | 7 | — | 2 | — | — | — | 5 | — | — | |
| IFNγ | — | 8542 | 83 | — | — | — | — | 137 | — | — | — | — | 57 | — | — |
| IL4 | — | 127 | 24 | — | — | — | 8 | 35 | — | — | — | — | 23 | — 11 | 44 |
| IL10 | — | 8551 | — | — | — | — | — | — | — | — | — | — | — | — | — |
| patient 067 | | | | | | | | | | | | | | | |
| IL2 | — | 1337 | 695 | 136 | 130 | 159 | — | 100 | — | — | — | — 85 | — 51 | — 76 | 60 |
| IFNγ | — | 2447 | 212 | — | — | — | — | — | — | — | — | — | — | — | — |
| IL4 | — | 632 | — | — | — | — | — | — | — | — | — | — | — | — | — |
| IL10 | — | 665 | — | — | — | 40 | — | 32 | 43 | 58 | 55 | — | — | 86 | 31* |
| patient 630 | | | | | | | | | | | | | | | |
| IL2 | — | 18874 | 55 | 82 | 2.4 | 3.4 | — | 12* | — | — | — | — | — | — | 2.4* |
| IFNγ | — | 474 | — | — | — | — | — | — | — | — | — | — | — | — | — |
| IL4 | — | 251 | — | — | — | — | — | — | — | — | — | — | — | — | — |
| IL10 | — | 242 | — | — | — | — | — | — | — | — | — | — | — | — | — |
| patient 078 | | | | | | | | | | | | | | | |
| IL2 | — | 207 | 48 | — | — | — 44 | — | — | — | — | | — 17 | | | |
| IFNγ | — | 2249 | — | — | — | — | — | — | — | — | — | — | — | — | — |
| IL4 | — | 55 | — | — | — | — | — | — | — | — | — | — | — | — | — |
| IL10 | — | 207 | — | 64 | — 109 | 85 | 64 | 16 | 41 | 58 | 43 | — | 54 | 21 | 38 — |
| patient 659 | | | | | | | | | | | | | | | |
| IL2 | — | 153 — | — | — | — | — | — | — | — | — | — | 18 | — | — | — |
| IFNγ | — | — 187 | — | — | — | — | — | — | — | — | — | — | — | — | — |
| IL4 | — | 453 | — | — | — | — | — | — | — | — | — | — | — | — | — |
| IL10 | — | 182 | — | 129 | 40 | — | 31 | — | — | 18 | — | — | 29 | — | — |

*assay at 24 h instead of 48 h; NT: not tested; —: not detected

Table X shows that 8 patients out of 9 have cells specific for at least one of C or NS3 peptides, which cells secrete IL-2, IFN-γ, IL-4 or IL-10. It also shows that a mixture of C and NS3 peptides makes it possible to detect HCV-specific T lymphocytes at least as effectively as with the peptides used separately, and that it even increases the sensitivity of this detection (see patient 342).

These results indicate that the peptides having good affinity for the HLA II molecules, as defined above, can be used in a diagnostic test for the immune state of patients with respect to HCV infection.

1-25. (canceled)

26. A peptide mixture comprising at least two different peptides derived from the hepatitis C virus (HCV), at least one of which is a peptide derived from the C protein that binds to at least four different HLA II molecules whose allelic frequency is greater than 5% in the Caucasian population with a binding activity of <1000 nM.

27. The peptide mixture as claimed in claim 26, further comprising at least one peptide derived from the NS3 protein that binds to at least four different HLA II molecules whose allelic frequency is greater than 5% in the Caucasian population with a binding affinity of <1000 nM.

28. The peptide mixture as claimed in claim 26, wherein said HLA II molecules are selected from the group of molecules consisting of HLA-DR1, HLA-DR3, HLA-DR4, HLA-DR7, HLA-DR11, HLA-DR13, HLA-DR15, HLA-DRB3, HLA-DRB4, HLA-DRB5 and HLA-DP4.

29. The peptide mixture as claimed in claim 27, wherein said HLA II molecules are selected from the group of molecules consisting of HLA-DR1, HLA-DR3, HLA-DR4, HLA-DR7, HLA-DR11, HLA-DR13, HLA-DR15, HLA-DRB3, HLA-DRB4, HLA-DRB5 and HLA-DP4.

30. The peptide mixture as claimed in claim 28, wherein said HLA II molecules are encoded, respectively, by the HLA alleles DRB1*0101, DRB1*0301, DRB1*0401, DRB1*0701, DRB1*1101, DRB1*1301, DRB1*1501, DRB3*0101, DRB4*0101, DRB5*0101, DP*0401 and DP*0402.

31. The peptide mixture as claimed in claim 29, wherein said HLA II molecules are encoded, respectively, by the HLA alleles DRB1*0101, DRB1*0301, DRB1*0401, DRB1*0701, DRB1*1101, DRB1*1301, DRB1*1501, DRB3*0101, DRB4*0101, DRB5*0101, DP*0401 and DP*0402.

32. The peptide mixture as claimed in claim 26, wherein the peptides derived from the C protein of HCV are selected from the group consisting of:
a) the peptides corresponding, respectively, to positions 19-47, 27-51, 31-57, 104-133 and 127-167 of the HCV protein,
b) the peptides comprising less than the entire sequence of the peptides as defined in a) and including at least 11 consecutive amino acids of the peptides as defined in a), and
c) the peptides derived from the peptides as defined in a) or in b) and further modified by substitution of cysteine residues for the alanine residues (C→A) at position +1 or +2, relative to the amino acid residue at the N-terminal position and/or at position −1, −2 or −3, relative to the amino acid residue at the C-terminal position.

33. The peptide mixture as claimed in claim 32, wherein the peptides of at least 11 amino acids as defined in b) are selected from the group consisting of:
the peptide included in peptide 27-51 that corresponds to positions 27-41,
the peptide included in peptide 31-57 that corresponds to positions 31-45, and
the peptides included in peptide 127-167 that correspond, respectively, to positions 127-149, 131-145, 131-148, 131-167, 134-148 and 148-167.

34. The peptide mixture as claimed in claim 32, wherein the peptides as defined in c) are selected from the group consisting of the peptide derived from the C peptide 127-149 (SEQ ID NO:5).

35. The peptide mixture as claimed in claim 27, wherein the peptides derived from the NS3 protein are selected from the group consisting of:
a) the peptides corresponding, respectively, to positions 1007-1037, 1036-1055, 1052-1072, 1076-1093, 1127-1153, 1149-1172, 1174-1195, 1190-1212, 1206-1239, 1246-1275, 1275-1304, 1361-1387, 1377-1403, 1404-1432, 1456-1481, 1495-1513, 1524-1553 and 1552-1583, of the HCV protein,
b) the peptides comprising less than the entire sequence of the peptides as defined in a) and including at least 11 consecutive amino acids of the peptides as defined in a), and
c) the peptides derived from the peptides as defined in a) or in b) and further modified by substitution of cysteine residues for the alanine residues (C→A) at position +1 or +2, relative to the amino acid residue at the N-terminal position, and/or at position −1, −2 or −3, relative to the amino acid residue at the C-terminal position.

36. The peptide mixture as claimed in claim 35, wherein the peptides of at least 11 amino acids as defined in b) are selected from the group consisting of:
the peptides included in peptide 1007-1037; that correspond, respectively, to positions 1007-1021, 1015-1029, 1015-1037, 1019-1033 and 1020-1034,
the peptides included in peptide 1174-1195 that correspond, respectively, to positions 1174-1188, 1174-1192 and 1178-1192,
the peptides included in peptide 1190-1212 10 that correspond, respectively, to positions 1190-1204 and 1192-1206,
the peptides included in peptide 1246-1275 that correspond, respectively, to positions 1246-1260, 1246-1264, 1250-1264 and 1261-1275,
the peptides included in peptide 1377-1403 that correspond, respectively, to positions 1381-1395, 1381-1397, 1381-1403 and 1383-1397,
the peptide included in peptide 1495-1513 that corresponds, respectively, to positions 1495-1509,
the peptides included in peptide 1524-1553 that correspond, respectively, to positions 1524-1552, 1524-1538, 1528-1542, 1528-1552, 1529-1543, 1534-1548, 1538-1552 and 1540-1553, and
the peptides included in peptide 1552-1583 I 25 that correspond, respectively, to positions 1559-1573 and 1563-1577.

37. The peptide mixture as claimed in claim 35, wherein the peptides as defined in c) are selected from the group consisting of the following sequences SEQ ID NO.: 10, SEQ ID NO 13, SEQ ID NO 20, SEQ ID NO 22 and SEQ ID NO 24 and the sequences derived from the sequence of SEQ ID NO:24 that correspond, respectively, to positions 1524-1538, 1524-1552, 1528-1552, 1538-1552 and 1540-1553 of the HCV protein.

38. The peptide mixture as claimed in claim 27, wherein said peptides include peptides derived from the C and NS3 proteins of the HCV genotype 1.

39. The peptide mixture as claimed in claim 27, wherein 2 to 6 different peptides derived from the C and NS3 proteins are included, all of which peptides bind to at least 10 different HLA II molecules whose allelic frequency is greater than 5% in the Caucasian population.

40. The peptide mixture as claimed in claim 39, wherein the peptides are selected from the group consisting of the peptides derived from the C protein that correspond, respectively, to positions 27-51, 131-167, 127-149, 131-148 and 148-167 and the peptides derived from the NS3 protein that correspond, respectively, to positions 1007-1037, 1015-1037, 1036-1055, 1174-1192, 1190-1212, 1246-1264, 1381-1403, 1381-1397, 1524-1553, 1528-1552 and 1552-1583 of the HCV protein.

41. The peptide mixture as claimed in claim 27, wherein the peptides are in the form of a single fusion protein comprising a sequence of the peptides of said mixture, with the exclusion of the sequence corresponding to the fusion of the peptides C 31-45, C 141-155 and NS3 1207-1221 of the HCV protein.

42. A nucleic acid molecule that encodes a fusion protein as claimed in claim 41.

43. A recombinant vector, comprising a nucleic acid molecule as claimed in claim 42.

44. A cell that has been transformed with a vector as claimed in claim 43.

45. An anti-HCV immunogenic composition, comprising at least one peptide mixture as claimed in claim 27 in combination with at least one pharmaceutically acceptable vehicle.

46. The anti-HCV immunogenic composition as claimed in claim 45 further comprising an adjuvant.

47. An anti-HCV immunogenic composition, comprising at least one nucleic acid molecule as claimed in claim 42 in combination with at least one pharmaceutically acceptable vehicle.

48. The anti-HCV immunogenic composition as claimed in claim 47 further comprising an adjuvant.

49. An anti-HCV immunogenic composition, comprising at least one recombinant vector as claimed in claim 43 in combination with at least one pharmaceutically acceptable vehicle.

50. The anti-HCV immunogenic composition as claimed in claim 49 further comprising an adjuvant.

51. The immunogenic composition as claimed in claim 45, wherein said peptides are selected from the group consisting of modified peptides, peptides associated with liposomes and peptides associated with lipids.

52. The immunogenic composition as claimed in claim 51, wherein said peptide mixture is combined:
with one or more peptides or lipopeptides containing one or more CD8+ epitopes,
with other peptides comprising multiple CD4+ epitopes and/or
with one or more peptides or lipopeptides containing one or more B epitopes.

53. The immunogenic composition as claimed in claim 52 wherein said CD8+ epitopes are selected from the group consisting of the C peptides 2-10, 28-36, 35-44, 41-49, 42-50, 85-98, 88-97, 127-140, 131-140, 132-140, 167-176, 178-187, 181-190; the E1 peptides 220-227, 233-242, 234-242, 363-371; the E2 peptides 401-411, 460-469, 489-496, 569-578, 621-628, 725-733; the NS2 peptides 826-838, 838-845; the NS3 peptides 1073-1081, 1169-1177, 1287-1296, 1395-1403, 1406-1415; the NS4A peptides 1585-1593, 1666-1675; the NS4B peptides 1769-1777, 1789-1797, 1807-1816, 1851-1859; the NSSA peptide 2252-2260 and the NS5B peptides 2588-2596 and 2727-2735 of the HCV protein.

54. The immunogenic composition as claimed in claim 52 wherein said CD4+ epitopes are selected from the group consisting of the tetanus toxin TT peptide (positions 830-846), the Influenza hemagglutinin HA peptide (positions 307-319), PADRE and the Plasmodium falciparum LSA3 peptide.

55. The immunogenic composition as claimed in claim 52 wherein said B epitopes are specifically recognized by antibodies directed against either the C peptide 5-27, the NS4 peptide 1698-1719 or the NS5 peptide 2295-2315 of the HCV protein.

56. A vaccine comprising an immunogenic composition as claimed in claim 45.

57. A peptide selected from the group consisting of:
the peptides derived from the C protein of HCV that bind to at least four different HLA II molecules whose allelic frequency is greater than 5% in the Caucasian population with a binding activity of <1000 nM, with the exclusion of the peptide C 31-45, 21-40, C 20-44, C 23-42, C 111-130, C 109-128, C 128-152, C 131-150, C 133-152, C 138-162, C 141-155, C 142-161, C 141-160 and C 145-164,
the peptides derived from the NS3 protein of HCV selected from the group consisting of peptides corresponding, respectively, to positions 1007-1037, 1036-1055, 1052-1072, 1076-1093, 1127-1153, 1149-1172, 1174-1195, 1190-1212, 1206-1239, 1275-1304, 1361-1387, 1377-1403, 1404-1432, 1456-1481, 1495-1513, 1524-1553 and 1552-1583,
the peptides of at least 11 consecutive amino acids included in the above peptides, with the exclusion of the peptides NS3 1384-1401 and NS3 1207-1221,
the peptides corresponding, respectively, to positions 1246-1260 and 1261-1275 of the HCV protein, and
the peptides derived from the above peptides modified by substitution of cysteine residues for the alanine residues (C→A) at positions +1 or +2, relative to the amino acid residue at the N-terminal position and/or at positions −1, −2 or −3, relative to the amino acid residue at the C-terminal position.

58. A diagnostic reagent comprising at least one peptide as claimed in claim 57 either labeled or complexed in the form of multimeric complexes.

59. A method for evaluating the immune state of an individual, comprising detecting the presence of CD4+ T cells specific for HCV peptides by administration of the diagnostic reagent of claim 58 followed by application of an appropriate mechanism adequate for detection of the label or complex.

60. A method for sorting HCV-specific T lymphocytes, comprising at least the following steps:
bringing a suspension of cells to be sorted into contact with one or more labeled tetramers formed from complexes of C and/or NS3 peptides of the HCV protein and with soluble HLA II molecules, and
separating the cells labeled with the tetramers from the unlabeled cells.

61. A method for evaluating the immune state of an individual, comprising detecting the presence of CD₄+T cells specific for HCV peptides by administration of a peptide as defined in claim 26 either labeled or complexed in the form of multimeric complexes followed by detection of the label or complex.

62. A method for evaluating the immune state of an individual, comprising detecting the presence of CD₄+T cells specific for HCV peptides by administration of a peptide as defined in claim 27 either labeled or complexed in the form of multimeric complexes followed by detection of the label or complex.

63. A method for evaluating the immune state of an individual, comprising detecting the presence of CD₄+T cells specific for HCV peptides by administration of a peptide as defined in claim 26 and detection of the complex formed between CD₄+T cells and said peptide.

64. A method for evaluating the immune state of an individual, comprising detecting the presence of CD₄+T cells specific for HCV peptides by administration of a peptide as defined in claim 27 and detection of the complex formed between CD₄+T cells and said peptide.
